(19) United States
(12) Patent Application Publication
(10) Pub. No.: US 2011/0006218 A1
(43) Pub. Date: Jan. 13, 2011

(54) NANODIAMOND COMPOSITIONS AND METHODS OF MAKING AND USING THEREOF

(75) Inventors: Vadym Mochalin, Philadelphia, PA (US); Yury Gogotsi, Warminster, PA (US); Gleb Yushin, Atlanta, GA (US); Kristopher Behler, Elkton, MD (US); Jameson Detweiler, Akron, PA (US); Adrian Gurga, Urbana, IL (US)

Correspondence Address: WOODCOCK WASHBURN LLP, CIRA CENTRE, 12TH FLOOR, 2929 ARCH STREET, PHILADELPHIA, PA, 19104-2891, US

(73) Assignee: Drexel University, Philadelphia, PA (US)

(21) Appl. No.: 12/665,849

(22) PCT Filed: Jun. 19, 2008
(86) PCT No.: PCT/US08/67479

§ 371 Date: May 7, 2010

Related U.S. Application Data
(63) Continuation of application No. 60/947,533, filed on Jul. 2, 2007.

(51) Int. Cl. G01J 1/58; C07C 11/00; C07K 17/14; C07H 21/00; C08F 112/08; C08F 120/06; C08F 114/18; C08F 110/02; C08F 120/44; C08F 110/06; C08G 18/00; C08G 64/00; C08G 65/04; H01B 1/24; B29C 47/00

(52) U.S. Cl. 250/459.1; 250/458.1; 585/16; 530/391.1; 530/402; 536/22.1; 526/346; 526/317.1; 526/242; 526/352; 526/341; 526/351; 528/85; 528/370; 528/421; 252/511; 264/465; 264/442

(57) ABSTRACT

Provided are functionalized nanodiamonds. Also provided are methods for fabricating such functionalized nanodiamonds. Also provided are composites including nanodiamonds and polymers. Also provided are methods for fabricating such composites including nanodiamonds and polymers. Also provided are electrospun fibers including nanodiamonds and polymers. Also provided are methods for fabricating such electrospun fibers including nanodiamonds and polymers.

### CROSS REFERENCE TO RELATED APPLICATIONS

The present application claims priority to U.S. Provisional Patent Application No. 60/947,533, entitled “MODIFIED ULTRADISPERSE DIAMONDS AS NON-TOXIC QUANTUM DOTS FOR BIOMEDICAL APPLICATIONS,” filed Jul. 2, 2007, which is hereby incorporated by reference in its entirety.

### FIELD OF THE INVENTION

The present invention pertains to the field of nanoscale materials. The present invention also pertains to the field of surface chemistry. The present invention also pertains to polymer-nanoparticle composite materials.

### BACKGROUND OF THE INVENTION

Semiconductor quantum dot nanoparticles are considered promising materials for biomedical labeling and imaging. Bright and size-dependent fluorescence enables multicolor cell and tissue imaging, aiding in the diagnosis of diseases, tracking of drug delivery within the body, and even enabling infrared-guided surgery.

At present, quantum dots are widely used in in vitro applications. Their use in in vivo applications, however, is hindered because of the presence in quantum dots of high levels of toxic heavy metals such as selenium (Se), cadmium (Cd), astatine (As) and lead (Pb). The toxicity of such semiconductor quantum dots can be reduced by encapsulating the toxic core materials with less toxic shell materials. Less-toxic alternatives to semiconductor quantum dots are currently being developed, which alternatives include fluorescent dye filled silica and polymer nanospheres.

Nanoparticles are also available in the form of nanodiamonds. Ultradispersed nanodiamonds produced by detonational synthesis typically have a narrow particle size distribution with a mean value of about 5 nm. Nanodiamonds typically comprise a nanosized diamond core sheathed in one or more shells of graphitic and amorphous carbon.

Nanodiamonds are typically biocompatible and are of low toxicity. However, existing methods for functionalizing nanodiamonds for use in biological applications involve gaseous halogens or halogen acids. E.g., U.S. Pat. App. No. 2006/0269467, published Nov. 30, 2006; U.S. Pat. App. No. 2005/0158549, published Jun. 21, 2005. Such halogen materials, however, pose safety and handling challenges.

Accordingly, there is a need for functionalized nanodiamonds that are useful for, among other things, applications including composite materials, alternatives in biological applications to semiconductor quantum dots, and other compositions comprising nanodiamonds. There is also an attendant need for methods of fabricating such functionalized nanodiamonds, without using materials that pose the safety and handling challenges of halogen gases or halogen acids.

### SUMMARY OF THE INVENTION

In meeting the described challenges, provided is a composition, comprising at least one functionalized nanodiamond, comprising at least one acyl group linked to one or more surface groups.

Also provided is a method for synthesizing a functionalized nanodiamond having at least one surface group, comprising reacting at least one nanodiamond with at least one donor species to give rise to at least one nanodiamond intermediate, the nanodiamond comprising a carboxylic acid group, an ester, or any combination thereof, the nanodiamond intermediate comprising at least one acyl-halogen group or at least one acyl-tosylate group, or both; reacting the at least one nanodiamond intermediate with at least one displacer group, the displacer group displacing either an acyl-bound halogen or a tosylate, or both, of the at least one nanodiamond intermediate with at least one functionality, so as to give rise to at least one functionalized nanodiamond.

Additionally disclosed is a nanodiamond tracer, comprising a functionalized nanodiamond comprising at least one functionality linked to a nanodiamond by an acyl linkage, the functionalized nanodiamond being capable of emitting a signal.

Further provided is a method for sensing a nanodiamond, comprising detecting at least one signal emitted by at least one nanodiamond comprising at least one acyl linkage covalently bonded to at least one functionality capable of generating a signal.

Also disclosed is a detector system, comprising at least one functionalized nanodiamond, the functionalized nanodiamond comprising at least one acyl linkage covalently bonded to at least one functionality capable of generating a signal; and a detector capable of detecting one or more signals generated by the nanodiamond.

Also disclosed is a method for synthesizing a polymer-nanodiamond composite, comprising reacting at least one nanodiamond with at least one donor species to give rise to at least one nanodiamond intermediate, the nanodiamond comprising a carboxylic acid group, an ester, or any combination thereof, the nanodiamond intermediate comprising at least one acyl-halogen group, at least one acyl-tosylate group, or both; reacting the at least one nanodiamond intermediate with at least one displacer group, the displacer group displacing an acyl-bound halogen, an acyl-bound tosylate, or both, of the nanodiamond intermediate with at least one first monomer, so as to give rise to at least one monomer-bearing nanodiamond; and polymerizing at least one monomer-functionalized nanodiamond with a second monomer to give rise to a polymer-nanodiamond composite.

Also disclosed are compositions of matter comprising at least one nanodiamond covalently bonded to a polymer.

Also disclosed are articles of manufacture comprising an electrospun fiber comprising a polymer and at least one nanodiamond.

Also disclosed are processes comprising electrospinning a mixture comprising a polymer and at least one nanodiamond.

The general description and the following detailed description are exemplary and explanatory only and are not restrictive of the invention, as defined in the appended claims. Other aspects of the present invention will be apparent to those skilled in the art in view of the detailed description of the invention as provided herein.

### BRIEF DESCRIPTION OF THE DRAWINGS

The summary, as well as the following detailed description, is further understood when read in conjunction with the appended drawings. For the purpose of illustrating the invention, there are shown in the drawings exemplary embodiments of the invention; however, the invention is not limited to the specific methods, compositions, and devices disclosed. In addition, the drawings are not necessarily drawn to scale. In the drawings:

FIG. 1 depicts one embodiment of a sample scheme for functionalizing a nanodiamond bearing a carboxylic acid group.

FIG. 2 illustrates infrared (“IR”) spectra of starting materials UD-90 nanodiamond and octadecylamine (“ODA”), and of functionalized UD-90 nanodiamonds.

FIG. 3 illustrates UD90 nanodiamond in toluene (left side of figure) and UD90-ODA functionalized nanodiamond in toluene (right side of figure).

FIG. 4 illustrates the fluorescence of octadecylamine-modified nanodiamond;

FIG. 4a illustrates (from left to right, all under ultraviolet (“UV”) irradiation (λ_{excitation}=365 nm)): dilute octadecylamine-modified nanodiamond in dichloromethane (UD90-ODA), ODA in dichloromethane, concentrated UD90-ODA solution in dichloromethane, and neat dichloromethane; FIG. 4b illustrates fluorescence spectra (λ_{excitation}=400 nm) of solutions of UD90-ODA, UD90 and ODA in dichloromethane and pure dichloromethane (octadecylamine-modified nanodiamond showed the strongest fluorescence).

FIG. 5 illustrates the fluorescence under UV light of nanodiamond UD90 linked to octadecylamine.

FIG. 6 illustrates a sequence of reactions yielding an amine-modified nanodiamond.

FIG. 7 illustrates FTIR spectra of initial purified nanodiamond (A) and aminated nanodiamond-ethylene diamine (C).

FIG. 8 illustrates FTIR spectra in amide bond vibrations area for initial purified nanodiamond (A) and aminated nanodiamond-ethylene diamine (C).

FIG. 9 illustrates several example structures for nanodiamond—polymer composites, including an example where the polymers and nanodiamonds are covalently attached, denoted supercomposite in the figure.

FIG. 10 illustrates a scheme for the synthesis of an epoxy polymer using aminated nanodiamond.

FIG. 11 illustrates a scheme for the synthesis of aminated nanodiamond (A), and a scheme for combining aminated nanodiamond with epoxide monomers (B) to form a nanodiamond poly(epoxide) (C).

FIG. 12 illustrates a poly(expoide), wherein the groups denoted “R” may represent nanodiamonds.

FIG. 13 illustrates a poly(epoxide) including nanodiamonds covalently attached.

FIG. 14 illustrates the displacement of several epoxide resins as a function of load, including indication of the amount of creep.

FIG. 15 illustrates a stress-strain observation for an epoxide polymer including 5 weight percent aminated nanodiamond.

FIG. 16 illustrates a stress-strain observation for an epoxide polymer.

FIG. 17 illustrates a stress-strain observation for an epoxide polymer including 5 weight percent nanodiamond.

FIG. 18 illustrates a stress-strain observation for an epoxide polymer including 5 weight percent aminated nanodiamond.

FIG. 19 illustrates a visually enhanced observation of scratches made in pure epoxide polymer (A) and in epoxide polymer including 5 weight percent aminated nanodiamond (B).

FIG. 20 illustrates penetration curves for a scratch test of an epoxide polymer illustrating pile-up, residual deformation, and maximum deformation.

FIG. 21 illustrates penetration curves for a scratch test of an epoxide polymer including 5 weight percent aminated nanodiamond, illustrating pile-up, residual deformation, and maximum deformation.

FIG. 22 illustrates a visually enhanced observation of electrospun fibers including polyacrylonitrile and nanodiamond.

FIG. 23 illustrates a visually enhanced observation of an electrospun fiber including polyacrylonitrile and nanodiamond, indicating a smooth surface and uniform distribution of nanodiamond.

FIG. 24 illustrates HRTEM and SEM (inset) images of electrospun fibers including polyacrylonitrile and 60 weight percent nanodiamond.

FIG. 25 illustrates a schematic of an electrospinning device.

FIG. 26 illustrates SEM (a) and TEM (b,c) images of electrospun fibers including acrylonitrile and 10 weight percent nanodiamonds; SEM (d) and TEM (e,f) images of electrospun fibers including acrylonitrile and 60 weight percent nanodiamond. The inset in (e) shows the brittle fracture of the fiber.

FIG. 27 illustrates optimal transmittance as a function of wavelength for electrospun fibers including acrylonitrile and 10, 40, and 50 weight percent nanodiamond.

FIG. 28 illustrates SEM images of electrospun fibers including polyamide-11 and varying loading of nanodiamond: (a) 2.5 weight percent, (b) 10 weight percent, (c) 20 weight percent and (d) 40 weight percent.

FIG. 29 illustrates load—displacement curves (a) and hardness and Young's modulus (b) of films made from electrospun fibers including polyamide-11 and varying loading of nanodiamond. Inset in panel (a) shows a polyamide-11—nanodiamond film with 20 weight percent of nanodiamond on a thin glass slide. Film thickness was 2.6±0.4 μm.

FIG. 30 illustrates electrospun fibers including polyamide-11 and nanodiamond before (a) and after (b) melting on the surface of a computer chip.

### DETAILED DESCRIPTION OF ILLUSTRATIVE EMBODIMENTS

The present invention may be understood more readily by reference to the following detailed description taken in connection with the accompanying figures and examples, which form a part of this disclosure. It is to be understood that this invention is not limited to the specific devices, methods, applications, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

It is to be appreciated that certain features of the invention which are, for clarity, described herein in the context of separate embodiments, may also be provided in combination in a single embodiment. Conversely, various features of the invention that are, for brevity, described in the context of a single embodiment, may also be provided separately or in any subcombination. Further, reference to values stated in ranges include each and every value within that range.

The present invention first provides compositions, including at least one functionalized nanodiamond having at least one acyl group linked to one or more surface groups. Typically, the one or more surface groups include hydrocarbon chains, an alkene, an alkyne, a monomer, an aromatic molecule, a nucleophile, a fluorescent species, an antibody, a ligand, an amine, an amino group, a thiol, a sulfur, an acid, a base, an alcohol, a monomer, a polymer, a metal, a ceramic, a protein, a nucleic acid, a biochemical, or any combination thereof.

Surface groups that are fluorescent species enable the visualization, under proper conditions, of nanodiamonds bearing such species. Monomers are considered particularly suitable surface groups for the reason that their use would, as discussed elsewhere herein, enable nanodiamonds bearing such monomers to participate in polymerizations and become incorporated into polymeric materials.

Functionalized nanodiamonds typically have a characteristic cross-sectional dimension in the range of from about 1 nm to about 50 nm. Suitable nanodiamonds may also have characteristic cross-sectional dimensions in the range of from about 5 nm to about 20 nm.

Suitable compositions can, in some embodiments, include a solvent. In such embodiments, at least one functionalized nanodiamond is dispersed in the solvent. Suitable solvents include toluene, benzene, dichloromethane, n-n dimethylformamide, acetone, ethanol, or any combination thereof. It will be apparent to those having ordinary skill in the art that the dispersion of nanodiamonds bearing a given surface group depends upon interactions between the solvents and surface groups. As a non-limiting example, nanodiamonds bearing alkyl chains may not disperse homogeneously within a solvent comprising primarily water. Because they do not contain heavy metals or other toxins, it is believed—without being bound to any one theory of operation—that certain configurations of the disclosed functionalize nanodiamonds are suitable for in vivo use.

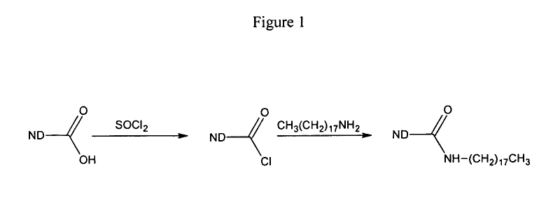

The present invention also discloses methods for synthesizing functionalized nanodiamonds having at least one functionality. An embodiment of these methods is shown schematically in FIG. 1 The methods include reacting at least one nanodiamond with at least one donor species to give rise to at least one nanodiamond intermediate. Suitable nanodiamonds are commercially available from Nanoblox, Inc. (www.nanobloxinc.com, Boca Ration, Fla., USA), and may be synthesized by a detonation process. Suitable nanodiamonds include a carboxylic acid group, an ester, or any combination thereof.

The nanodiamond intermediates of the methods described herein may include at least one acyl-halogen group or at least one acyl-tosylate group. These groups may result from the conversion by the donor species of the carboxylic acid groups or ester groups on the nanodiamonds, as shown in FIG. 1. Suitable donor species include a halogen-donating species, such as SOX₂ or PX₃, where X is a halogen. Thionyl chloride (SOCl₂) (available from., e.g., J. T. Baker, www.jtbaker.com, Phillipsburg, N.J., USA), is one suitable halogen-donating species. Tosylate-donating species are also suitable donor species.

The methods also include reacting the at least one nanodiamond intermediate with at least one displacer group. In this reaction, also illustrated in FIG. 1, the displacer group replaces either an acyl-bound halogen or a tosylate, or both, of the at least one nanodiamond intermediate with at least one functionality, so as to give rise to at least one functionalized nanodiamond.

Suitable displacer groups include molecules having an amino group, an alcohol, an amide, an aminoacid, a peptide, a hydroxyl group, a peptide, a protein, or any combination thereof. The displacer groups may also include a hydrocarbon chain, an aromatic group, a nucleophile, a fluorescent species, an amino group, a thiol, a sulfur, an acid, a base, a ligand, an antibody, a hydroxyl group, a protein, a biological molecule, a monomer, a nucleic acid, a polymer, a metal, an alcohol, or any combination thereof. As a non-limiting example, octadecylamine is a suitable displacer group. Other suitable displacer groups include alkyl lithium compounds, Grignard reagents, and the like. A particularly desired displacer group will depend on the user's ultimate needs and will be apparent to those having ordinary skill in the art.

Typically, nanodiamonds featured in the disclosed methods have a characteristic cross-sectional dimension in the range of from about 1 to about 10 nm. In some embodiments, two or more nanodiamonds are agglomerated into one or more particles having a characteristic cross-sectional dimension in the range of from about 20 nm to about 500 nm, or, in other embodiments, are agglomerated into one or more particles having a characteristic cross-sectional dimension in the range of from about 100 nm to about 200 nm.

The functionalized nanodiamonds of the disclosed methods typically have a characteristic cross-sectional dimension in the range of from about 1 nm to about 50 nm, or in the range of from about 10 nm to about 40 nm. The functionalized nanodiamonds may, in some embodiments, agglomerate into particles having a characteristic cross-sectional dimension in the range of from about 20 nm to about 500 nm. Functionalized nanodiamonds made according to the disclosed methods are also within the scope of the invention.

Also provided are nanodiamond tracers. These tracers include a functionalized nanodiamond comprising at least one functionality linked to the nanodiamond by an acyl linkage. Functionalities suitable for linking to the nanodiamond by an acyl linkage include hydrocarbon chains, aromatic groups, nucleophiles, fluorescent species, amino groups, thiols, sulfurs, acids, bases, proteins, biological molecules, monomers, polymers, metals, alcohols, radioactive species, magnetic species, or any combination thereof. Fluororophores are considered especially suitable functionalities, as are magnetic species.

Typically, the functionalized nanodiamond is capable of emitting a signal, including a visual signal, an infrared signal, an ultraviolet signal, a radioactive signal, a magnetic signal, an electrical signal, an electromagnetic signal, or any combination thereof. In some embodiments, the nanodiamond tracer emits one or more signals when illuminated by visible light, ultraviolet light, infrared light, x-rays, gamma rays, electromagnetic radiation, radio waves, radioactive particles, or some combination thereof. As a non-limiting example, a nanodiamond tracer bearing a fluorophore emits a fluorescent signal when the tracer is illuminated by light of a wavelength capable of exciting the fluorophore to emission.

In some embodiments, a nanodiamond tracer is capable of emitting a signal without necessarily being illuminated. As a non-restrictive example, a nanodiamond tracer bearing a magnetic species emits a magnetic signal without being illuminated by light or by an electric field. Similarly, a nanodiamond tracer bearing a radioactive species emits radioactive particles or waves without also necessarily being illuminated.

Functionalized nanodiamond tracers typically have a characteristic cross-sectional dimension in the range of from about 1 nm to about 50 nm, or in the range of from about 10 nm to about 30 nm. In some embodiments, two or more functionalized nanodiamond tracers agglomerate into particles having a characteristic cross-sectional dimension in the range of from about 20 nm to about 500 nm. Because certain embodiments of the nanodiamond tracers do not contain heavy metals or other toxins, it is believed—without being bound to any one theory of operation—that certain tracer embodiments are suitable for in vivo use.

Further disclosed are methods for sensing nanodiamonds. These methods include detecting at least one signal emitted by at least one nanodiamond comprising at least one acyl linkage covalently bonded to at least one functionality capable of generating a signal. Suitable functionalities are described elsewhere, and include, inter alia, hydrocarbon chains, aromatic species, fluorescent dyes, fluorescent proteins, heterocyclic compounds, magnetic molecules, radioactive species, or any combination thereof.

In some embodiments, the nanodiamond is illuminated with visible light, ultraviolet light, infrared light, an x-ray, a gamma ray, a radioactive particle, an electromagnetic wave, an electric field, or any combination thereof. In some of these embodiments, the illumination elicits one or more signals from the nanodiamond, such as a visual signal, an infrared signal, an ultraviolet signal, a radioactive signal, a magnetic signal, or any combination thereof. In other embodiments, the nanodiamond is not illuminated or otherwise excited, and the detected signal is one inherently emitted by the functionality bound to the nanodiamond. Non-limiting examples of such signals include magnetic signals, and radioactive signals. The signals may also be generated by absorption or transmission of any type of electromagnetic radiations.

Suitable nanodiamonds have a characteristic cross-sectional dimension in the range of from about 1 nm to about 50 nm, or in the range of from about 10 nm to about 30 nm. Within these ranges, nanodiamonds may also have a characteristic cross-sectional dimension of at least about 12 nm, or at least about 14 nm, or at least about 16 nm, or at least about 18 nm, or at least about 20 nm, or at least about 22 nm, or at least about 24 nm, or at least about 26 nm. Suitable nanodiamonds may also have a characteristic cross-sectional dimension in the range of from about 1 nm to about 10 nm. Within this range, nanodiamonds may also have a characteristic cross-sectional dimension of at least about 2 nm, or at least about 3 nm, or at least about 4 nm, or at least about 5 nm, or at least about 6 nm, or at least about 7 nm, or at least about 8 nm, or at least about 9 nm. The nanodiamonds may, in some embodiments, also agglomerate into particles having a characteristic cross-sectional dimension in the range of from about 20 nm to about 500 nm.

The signals are detected using a variety of methods. These methods include visually inspecting, monitoring electromagnetic radiation, monitoring radioactive emissions, monitoring a magnetic signal, or any combination thereof. As a non-limiting example, the presence of fluorophore-bearing nanodiamonds could be determined by illuminating a sample suspected of containing one or more such nanodiamonds with light known to be capable of exciting the fluorophores and inspecting the sample for the presence of excited fluorphores. Such an inspection could be performed by a microscope

Also disclosed are detection systems. These systems include at least one functionalized nanodiamond, the functionalized nanodiamond comprising at least one acyl linkage covalently bonded to at least one functionality capable of generating a signal; and a detector capable of detecting one or more signals generated by the nanodiamond.

Suitable functionalities capable of generating signals are described elsewhere herein. Such functionalities include, inter alia, a hydrocarbon chain, an aromatic, a fluorescent dye, a fluorescent protein, a heterocyclic compound, a magnetic molecule, a ligand, an antibody, a radioactive atom, or any combination thereof. In some embodiments, the functionality can be characterized as being capable of preferentially binding to one or more specific cells of an organism, preferentially binding to one or more specific materials, preferentially binding to one or more molecules of an organism, or any combination thereof. In one non-limiting example, a functionality on the nanodiamond would permit the nanodiamond to bind selectively to a particular molecule of interest in a sample, such as a cancerous cell. Such a nanodiamond could also include a fluorophore. By interrogating the sample for the nanodiamond's fluorescence, a user could then determine the presence of cancerous cells in the sample.

The detection systems typically include an excitation source, which sources suitably emit visible light, ultraviolet radiation, x-rays, magnetic waves, infrared light, microwaves, radio waves, or any combination thereof. Suitable excitation sources are capable of eliciting one or more signals from the at least one functionalized nanodiamond; such signals are described elsewhere herein. Excitation systems are not necessary in all embodiments—as described elsewhere herein, certain functionalized nanodiamonds can emit or absorb signals without excitation.

Functionalized nanodiamonds can have a characteristic cross-sectional dimension in the range of from about 1 nm to about 50 nm, or in the range of from about 10 nm to about 30 nm. In some embodiments, the functionalized nanodiamonds agglomerate into particles having a characteristic cross-sectional dimension in the range of from about 20 nm to about 500 nm. As discussed elsewhere herein, it is believed—without being bound to any one theory of operation—that some configurations of the disclosed functionalize nanodiamonds that do not contain heavy metals or toxins are suitable for in vivo use.

Also disclosed are methods for synthesizing polymer-nanodiamond composites. These methods include reacting at least one nanodiamond with at least one donor species to give rise to at least one nanodiamond intermediate, the nanodiamond comprising a carboxylic acid group, an ester, or any combination thereof, where the nanodiamond intermediate includes at least one acyl-halogen group, at least one acyl-tosylate group, or both. The methods may also include reacting the at least one nanodiamond intermediate with at least one displacer group. The displacer group suitably displaces an acyl-bound halogen, an acyl-bound tosylate, or both, of the nanodiamond intermediate, and replaces the displaced atom with at least one first monomer, so as to give rise to at least one monomer-bearing nanodiamond. The methods may also include polymerizing the monomer-bearing functionalized nanodiamond with a second monomer to give rise to a polymer-nanodiamond composite. Polymerization may be performed under conditions appropriate to the polymer being formed; certain catalysts and other reagents may be necessary to perform the polymerization, all of which will be apparent to those having ordinary skill in the art.

Suitable donor species are described elsewhere herein; the donor species convert a carboxylic acid group, an ester group, or both, of the at least one nanodiamond to an acyl-halogen group, an acyl-tosylate group, or both. Halogen-donating species can include SOX₂, PX₃ or any combination thereof, wherein X is a halogen. As described elsewhere herein SOCl₂ is a suitable halogen-donating species, as are SOBr₂ and SOI₂. Phosphorous trichloride, phorphorous tribromide, and phosphorous triiodide, are also suitable halogen donating species.

In typical embodiments, either the monomer-linked nanodiamond, the second monomer, or both, comprises an alkene, an alkyne, a styrene, an amide, an alcohol, an amino acid, an ester, or any combination thereof. Other suitable monomers will be apparent to those having ordinary skill in the art; the optimum monomer will depend on the user's needs.

Suitable displacer groups are described elsewhere herein, and can include, e.g., alkyl lithium species, amides, hydrides, hydroxyls, and the like. As one non-limiting example, amino-butylene would be a suitable displacer group.

In some embodiments, the monomer-linked nanodiamond and the second monomer have the same composition. In one non-limiting example, the first and second monomers would both be ethylene. In other embodiments, the monomer-linked nanodiamond and the second monomer have different compositions. This in turn enables incorporation of the nanodiamond into a copolymer. As a non-limiting example, a nanodiamond bearing a vinyl acetate could be polymerized with ethylene so as to form an ethylene vinyl acetate copolymer that incorporates the nanodiamond. In copolymer embodiments of the present invention, the first and second monomers are chosen to achieve the desired copolymer; optimum monomers will be apparent to those having ordinary skill in the art.

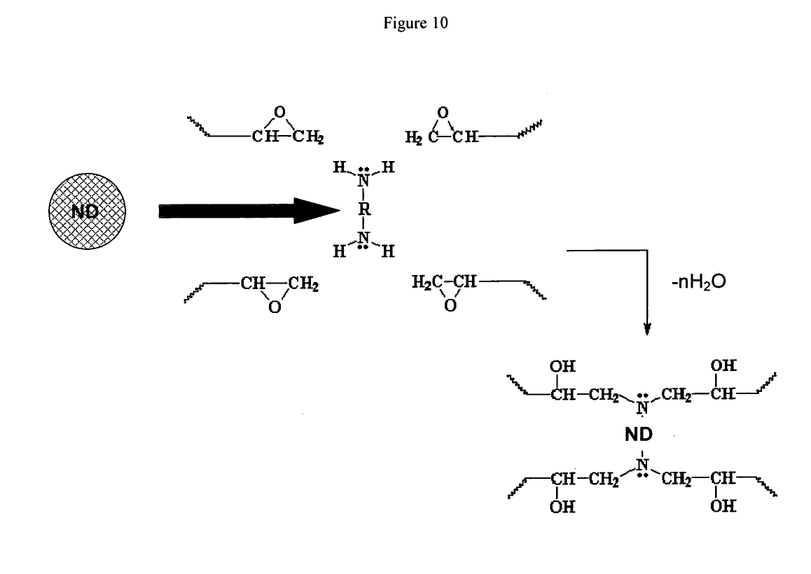

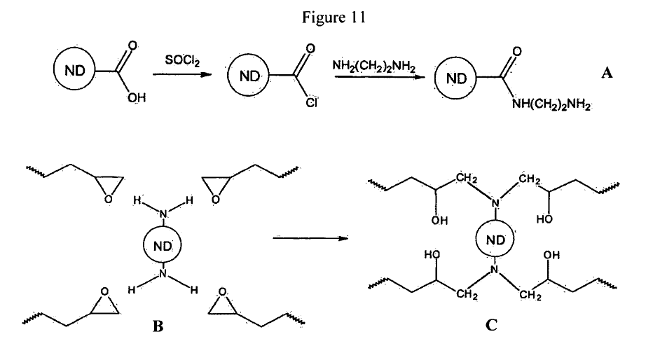

In certain embodiments, the first monomer may include an amine. In other embodiments, the second monomer may include an epoxide. In still further embodiments, the polymerizing may give rise to a poly(epoxide) compound. For example, nanodiamonds may be covalently attached to a first monomer including an amine, and polymerization with a second monomer including an epoxide may give rise to a poly(epoxide) polymer. An example synthetic scheme giving rise to an epoxide polymer including nanodiamonds is depicted in FIG. 10 and FIGS. 11(B and C).

Characteristic cross-sectional dimensions for nanodiamonds suitable for the present methods are described elsewhere herein. As also discussed elsewhere herein, in some embodiments nanodiamonds are agglomerated into particles having a characteristic cross-sectional dimension in the range of from about 20 nm to about 500 nm. The present invention also includes polymer-nanodiamond composites made according to the claimed methods.

Also disclosed are compositions of matter comprising at least one nanodiamond covalently bonded to a polymer.

In some embodiments, the polymer may comprise two or more monomer subunit species, where at least one of the monomer subunit species is covalently bonded to the at least one nanodiamond. For example, the polymer may comprise two monomer subunits, denoted A and B. The polymer may, for example, be formed of the A and B monomer subunits in an alternating fashion, e.g., ABAB, or in block fashion, e.g., AABB, branching fashion, multiple branching fashion, crosslinked network fashion, or other fashions. In some examples, the nanodiamond may be covalently bonded to monomer subunit A, or to monomer subunit B, or to both. For example, the nanodiamond may be covalently bonded only to monomer subunit A, denoted A-ND. The polymer may then, for example, be formed of the A-ND and B monomer subunits in an alternating fashion, e.g., (A-ND)B(A-ND)B. In some examples, any particular nanodiamond may be bonded to several monomer subunits of the same type, and in other examples, any particular monomer subunit may be bonded to several nanodiamonds. In circumstances where multiple monomer subunits are attached to each nanodiamond, polymerization may result in a highly branched or interconnected polymer matrix.

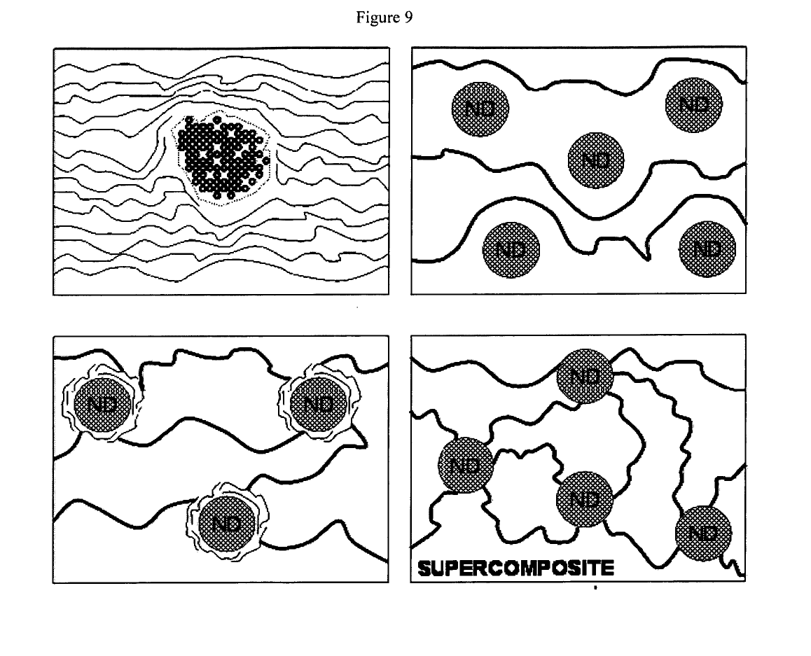

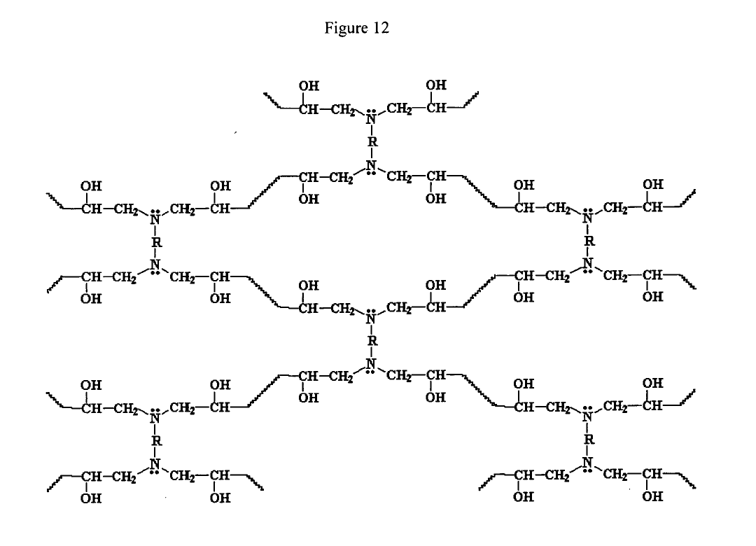

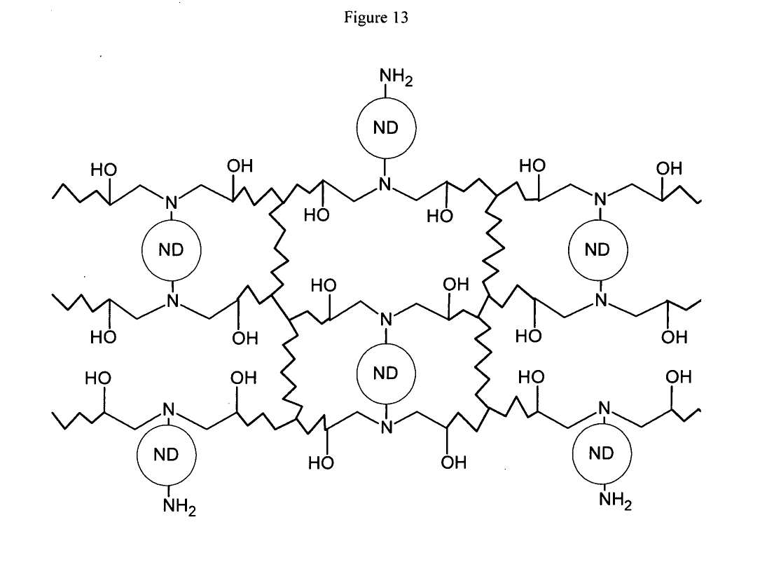

In further examples, there may be three, four, or more different monomer subunits. The polymer may be a sequence-specific heteropolymer, a block copolymer, or other structured polymer species. In some examples, one subset of types of monomer subunits may be bonded to nanodiamonds, and another subset of types of monomer subunits may not be bonded. For example, the composition may be depicted by the box denoted supercomposite in FIG. 9, indicating that the nanodiamonds are covalently attached to the polymer. In further examples, the structure of the polymer-nanodiamond composition may be depicted by FIG. 11(C), FIG. 12, or FIG. 13.

The compositions disclosed herein may include polymers comprising at least one of a styrenic polymer, an acrylic polymer, a fluoropolymer, a poly(epoxide), a poly(amide), a poly(ester), a poly(methylmethacrylate), a poly(ethylene), a poly(acrylonitrile), a poly(propylene), a thermoplastic poly(urethane), a poly(imide), a alkylene tetrafluoroethylene, a polycarbonate, a poly(ethylene oxide), a poly(caprolactone), or any copolymer or combination thereof.

The compositions disclosed herein may have one or more monomer subunits. For example, at least one monomer subunit species may include at least one of an amine, an alkene, an alkyne, a styrene, an amide, an alcohol, an amino acid, an ester, or any combination thereof. For example, one or at least one monomer subunit species may comprise at least one amine. For example, monomer subunit species comprising at least one amine may be the only one of the monomer subunit species that is covalently bonded to the at least one nanodiamond.

Nanodiamonds for use in the compositions disclosed herein can have a characteristic cross-sectional dimension in the range of from about 1 nm to about 50 nm, or in the range of from about 10 nm to about 30 nm, or in the range of from about 5 nm to about 20 nm.

Compositions disclosed herein may, for example, have a weight percent of nanodiamonds in the composition in the range from about 0.01 percent to about 90 percent based on total weight of the composition, or in the range from about 0.01 percent to about 25 percent based on total weight of the composition, or in the range from about 0.5 percent to about 1 percent based on total weight of the composition, or in the range from about 0.01 percent to about 5 percent based on total weight of the composition.

As disclosed elsewhere herein, the nanodiamonds in the compositions disclosed herein may include at least one nanodiamond produced by detonation synthesis.

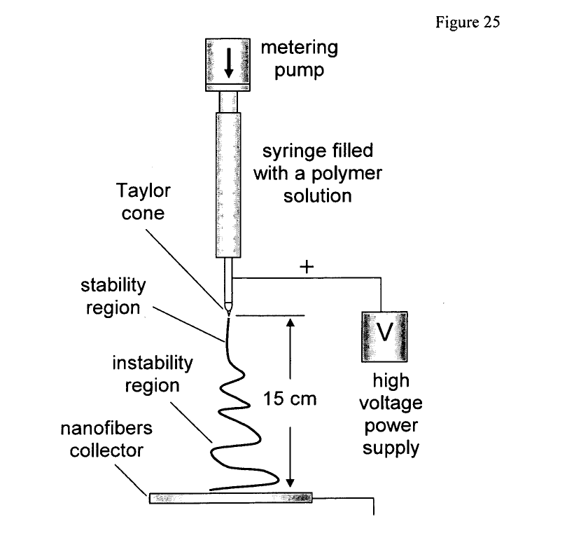

Also disclosed are articles of manufacture comprising electrospun fibers comprising a polymer and at least one nanodiamond. Electrospinning is a process whereby an electrical charge is used to draw fibers from a liquid (FIG. 25). Electrospinning provides certain benefits stemming from the small and tunable fiber diameter. Polymer composites produced via the electrospinning method allow for a polymer nanofiber to act as a host for nanoparticle material.

Polymer nanofibers may be used as a coating or appliqués, thus delivering nanodispersed particles while effectively preventing their agglomeration. Without being bound to any particular theory, the confinement of the fiber diameter, polymer surface tension and strong electrostatic force pulling the fiber in the electrospinning process may help in deagglomeration of nanoparticles. In addition, as soon as the fiber solidifies upon evaporation of the solvent during electrospinning, reagglomeration of nanoparticles may be effectively suppressed; thus, a resulting nanocomposite incorporates uniformly dispersed, size-confined nanoparticles.

Polymers suitable for electrospun fibers may include, for example, at least one of a styrenic polymer, an acrylic polymer, a fluoropolymer, a poly(epoxide), a poly(amide), a poly(ester), a poly(methylmethacrylate), a poly(ethylene), a poly(acrylonitrile), a poly(propylene), a thermoplastic poly(urethane), a poly(imide), a alkylene tetrafluoroethylene, a polycarbonate, a poly(ethylene oxide), a poly(caprolactone), or any copolymer or combination thereof.

Nanodiamonds for use in the articles disclosed herein can have a characteristic cross-sectional dimension in the range of from about 1 nm to about 50 nm, or in the range of from about 10 nm to about 30 nm, or in the range of from about 5 nm to about 20 nm.

Articles disclosed herein may, for example, have a weight percent of nanodiamonds in the electrospun fiber in the range from about 0.01 percent to about 90 percent based on total weight of the electrospun fiber, or in the range from about 10 percent to about 60 percent based on total weight of the electrospun fiber, or in the range from about 15 percent to about 30 percent based on total weight of the electrospun fiber.

As disclosed elsewhere herein, the nanodiamonds in the articles disclosed herein may include at least one nanodiamond produced by detonation synthesis.

Articles comprising eletrospun fibers may include nanodiamonds wherein at least one nanodiamond is a functionalized nanodiamond. For example at least one nanodiamond may be covalently bonded to the polymer. As disclosed elsewhere herein, the functionalization may take a variety of forms and may include covalent attachment to various monomer subunits, thus yielding, for example, polymers covalently attached to nanodiamonds. Composite polymers including covalently attached nanodiamonds may also be subject to electrospinning.

Also disclosed herein are processes comprising electrospinning a mixture comprising a polymer and at least one nanodiamond. In some instances, the mixture of polymer and at least one nanodiamond may be sonicated prior to, or in addition to, electrospinning.

Polymers suitable for an electrospinning process may include at least one of a styrenic polymer, an acrylic polymer, a fluoropolymer, a poly(epoxide), a poly(amide), a poly(ester), a poly(methylmethacrylate), a poly(ethylene), a poly(acrylonitrile), a poly(propylene), a thermoplastic poly(urethane), a poly(imide), a alkylene tetrafluoroethylene, a polycarbonate, a poly(ethylene oxide), a poly(caprolactone), or any copolymer or combination thereof.

Nanodiamonds for use in processes disclosed herein can have a characteristic cross-sectional dimension in the range of from about 1 nm to about 50 nm, or in the range of from about 10 nm to about 30 nm, or in the range of from about 5 nm to about 20 nm.

Processes disclosed herein may, for example, involve electrospinning mixtures having a weight percent of nanodiamonds in the mixture in the range from about 0.01 percent to about 90 percent based on total weight of the electrospun fiber, or in the range from about 10 percent to about 60 percent based on total weight of the electrospun fiber, or in the range from about 15 percent to about 30 percent based on total weight of the electrospun fiber.

As disclosed elsewhere herein, the nanodiamonds used in the processes disclosed herein may include at least one nanodiamond produced by detonation synthesis.

Electrospinning processes may make use of functionalized nanodiamonds. As disclosed elsewhere herein, functionalization may take a variety of forms and may include covalent attachment to various monomer subunits, thus yielding, for example, polymers covalently attached to nanodiamonds. Electrospinning processes may make use of a polymer and nanodiamonds wherein at least one nanodiamond is covalently bonded to the polymer. As described elsewhere herein, nanodiamonds may be covalently attached to polymers in a variety of ways, including attaching nanodiamonds to certain monomer subunits and thereafter forming a polymer using, among other species, the nanodiamond-bearing monomer subunits.

In some embodiments of the electrospinning processes, the process may further include fusing the polymer and the at least one nanodiamond. For example, the product of electrospinning may be then subjected to heat, causing the composite to melt and fuse, thereby, for example, coating a surface.

Various compositions and articles may be made according to the electrospinning processes disclosed herein, for example electrospun fibers, melted or fused electrospun fibers, coatings, and the like.

### EXAMPLES AND OTHER ILLUSTRATIVE EMBODIMENTS

The following are non-limiting examples and embodiments that are representative only and do not necessarily restrict the scope of the present invention.

### Example 1

Chemical linking of amino (NH₂)—terminated molecules was performed using wet chemistry approach through the forming of reactive intermediates—acyl chlorides by the reaction of nanodiamond with SOCl₂. A representative reaction scheme for the inventive process is shown in FIG. 1.

Initial material was nanodiamond UD90 produced by Federal Research and Production Center “Altai” (Russia) supplied by NanoBlox Inc. (USA). Prior to modification, initial UD90 powder was first rinsed with 10% aqueous HCl to convert anhydro- and carboxylate groups into carboxylic groups. 300 mg of UD90 powder were rinsed and dried at 120° C. and then refluxed with 50 ml of SOCl₂ (Alfa Aesar) and 1 ml of anhydrous dimethylformamide (used as catalyst) at 70° C. for 24 hours.

After removing the supernatant, the solid precipitate was washed with anhydrous tetrahydrofuran, centrifugated and decanted. This sequence of operations was continued until the supernatant became colorless. The remaining solid was dried at ambient temperature under vacuum.

The solid was then heated with 1 g of octadecylamine (ODA) at 90-100° C. for 96 hours. After cooling, excess octadecylamine was removed by sonication in ethanol. Remaining ethanol was evaporated at ambient temperature and the resultant dry powder was used for subsequent characterization.

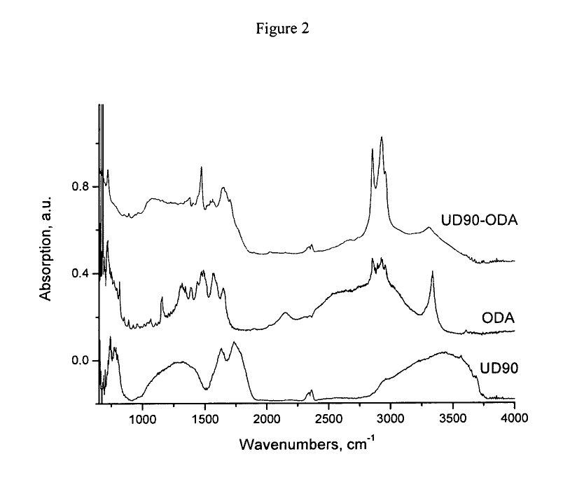

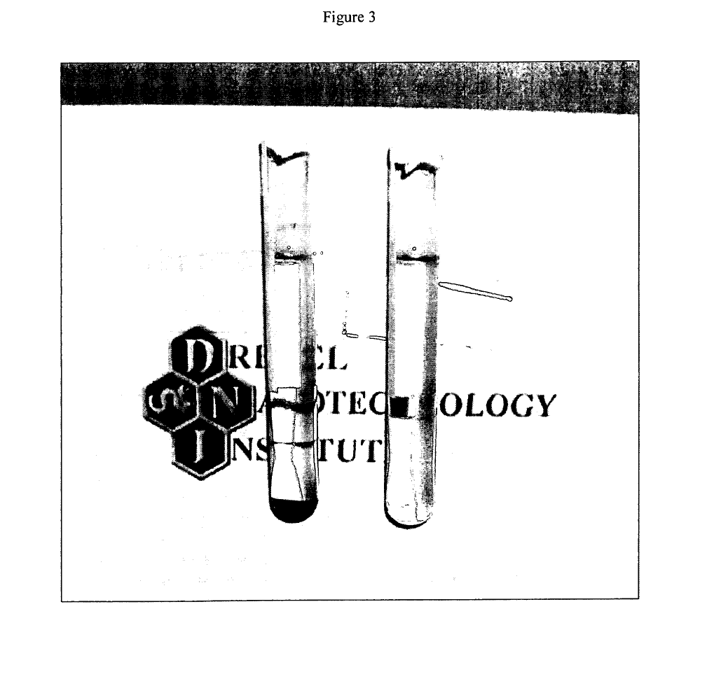

FTIR spectra of the product UD90-ODA material (FIG. 2) indicated long hydrocarbon chains, N—H vibrations, and a shift in position of C═O vibrations in the UD90-ODA, which evidences formation of the amide bond between the nanodiamond's COOH groups and the amino groups of ODA. Because of comparatively long hydrocarbon chains covalently bonded to the surface of the nanodiamond, UD90-ODA is a highly hydrophobic material which does not dissolve in water, dissolves poorly in polar organic solvents such as N,N-dimethylformamide, acetone, ethanol, and dissolves readily in non-polar or slightly polar organic solvents such as benzene, toluene, chloroform and dichloromethane (FIG. 3). The value of solubility of UD90-ODA was estimated gravimetrically as about 4 g/L in dichloromethane and about 3 g/L in toluene.

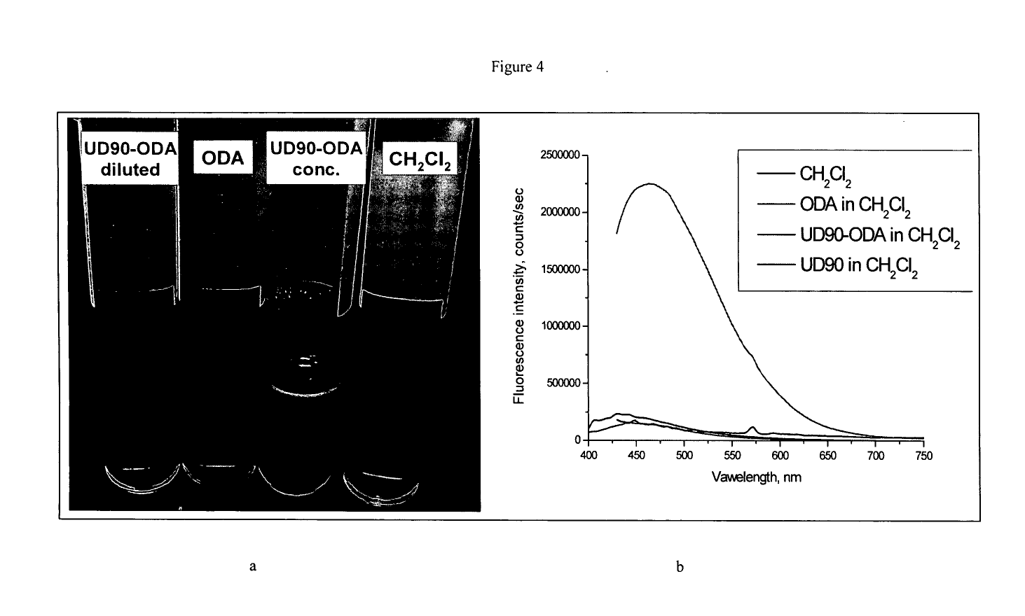

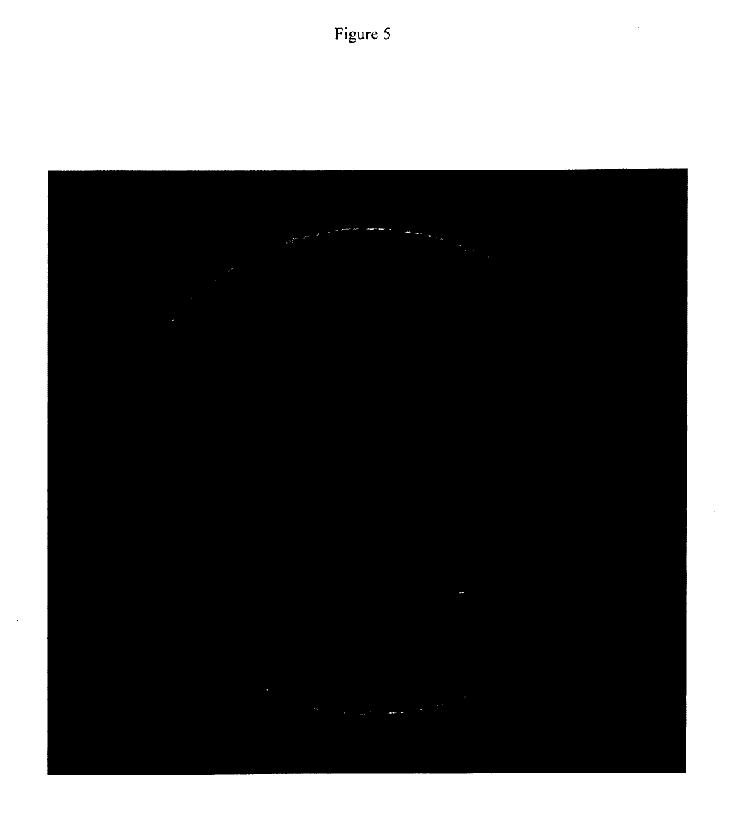

Additionally, the resulting UD90-ODA material exhibited a high level of fluorescence in solution, which fluorescence was visible under UV radiation at 365 nm wavelength. (FIG. 4a). Accordingly, the UD90-ODA material displayed quantitatively greater fluorescence than ODA, UD90, or dichloromethane (FIG. 4b). FIG. 5 illustrates qualitatively the fluorescence of UD90-ODA material under UV light. Thin layer chromatography was used for separation of fluorescent UD90-ODA from excess of reagents and by-products of wet chemistry process. Strong blue fluorescence was found for UD90-ODA upon UV-light illumination

### Example 2

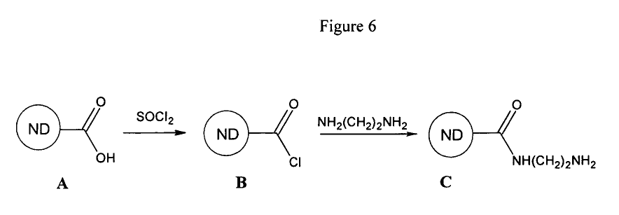

The sequence of reactions yielding the NH₂-terminated product—ND-EDA (Ethylenediamine) is illustrated in FIG. 6.

Nanodiamond powder UD90 produced by detonation synthesis (FRPC “Altai”, Russia) was supplied by NanoBlox, Inc., USA. The powder was purified by oxidation in air and then boiled with 35% wt. aqueous HCl under reflux for 24 h to remove traces of metals and metal oxides. After removing the excess of HCl ND powder was rinsed with DI water to neutral pH and dried in the oven at 110° C. overnight. This purified material (FIG. 6A) was used in subsequent functionalization.

The reagents employed, without additional purification, were thionyl chloride purum. ≧99.0% (Fluka), methanol anhydrous 99.8% (Sigma-Aldrich), tetrahydrofurane 99.85% ExtraDry (Acros Organics), ethylenediamine SigmaUltra (Sigma-Aldrich), and N,N-dimethylformamide anhydrous, 99.8%.

Synthesis of ND acylchloride derivative (FIG. 6B): To ˜1.5 g of A in round-bottom 100 ml flask with a Teflon-coated magnetic stirrer bar 50 ml of SOCl₂ and 0.5 ml of anhydrous dimethylfomamide (DMF) (catalyst) were added. The flask was closed with a stopper and sonicated in an ultrasound bath until there were no visible chunks of nanodiamond. Then the flask was connected to a reflux condenser closed with a desiccating tube (Drierit) and heated under refluxing at 70° C. during 24 h. After cooling down to room temperature an excess of SOCl₂ was removed by vacuum distillation at temperature ≦50° C. to prevent its decomposition. Then the content of the flask was rinsed with anhydrous THF (2 rinses 50 ml THF each), ND powder was let to precipitate and excess THF was removed by decantation. The flask was transferred into a desiccator with Drierit and left under vacuum at room temperature overnight to dry the powder (FIG. 6B).

Synthesis of ND amino derivative (FIG. 6C): ˜1.5 g of product (FIG. 6B) was mixed with 50 ml of anhydrous EDA in round-bottom 100 ml flask having a Teflon-coated magnetic stirrer bar in it. The flask was closed with a stopper and sonicated in an ultrasound bath at 60° C. for 5 h. After this the flask was connected to a reflux condenser closed with a desiccating tube (Drierit) and heated under refluxing at 60° C. for 24 h. After cooling down to room temperature and precipitation of the powder an excess of EDA was removed by centrifugation and the powder was successively rinsed with anhydrous methanol and centrifuged until neutral pH to wash out the adsorbed EDA. Resulting material was transferred onto a watch glass and dried at room temperature.

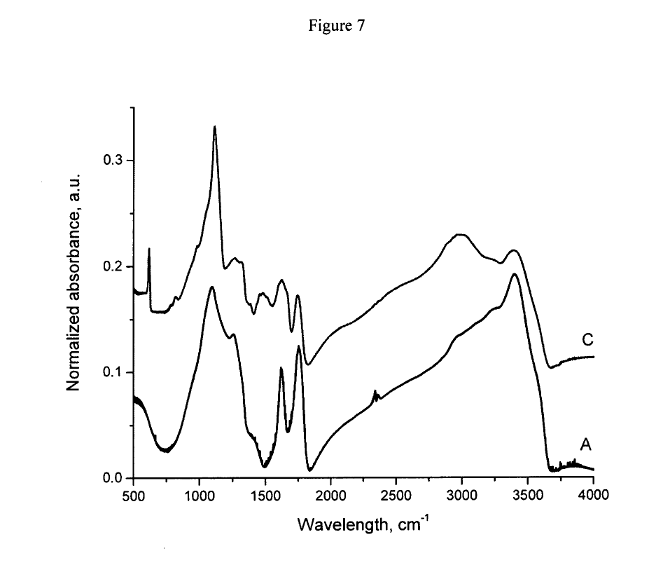

FTIR spectra of materials A (FIG. 6A) and C (FIG. 6C) were recorded in KBr pellets using Digilab FTIR Spectroscope. The FTIR spectra of the initial purified ND, A, and aminated ND-EDA, C, are illustrated in FIG. 7.

N—H stretching vibrations area strongly overlaps with O—H stretching thus the peak at 3300 cm⁻¹ in FIG. 7 can be assigned to O—H stretch in A and N—H and O—H stretch in C. Increased aliphatic C—H stretch intensity in C (2850-3000 cm⁻¹) is an indicative of EDA hydrocarbon chains which can be however both chemically linked and/or physically sorbed at the surface of nanodiamond in C. Strong band at 1110 cm⁻¹ in C can be assigned to C—N stretching vibrations but this assignment is not less reliable because in this area—fingerprints area—peak assignment is problematic. Nevertheless the presence of C—N bonds with corresponding stretching vibrations at 1080-1140 cm⁻¹ is not in contradiction with the sequence of reactions in FIG. 6.

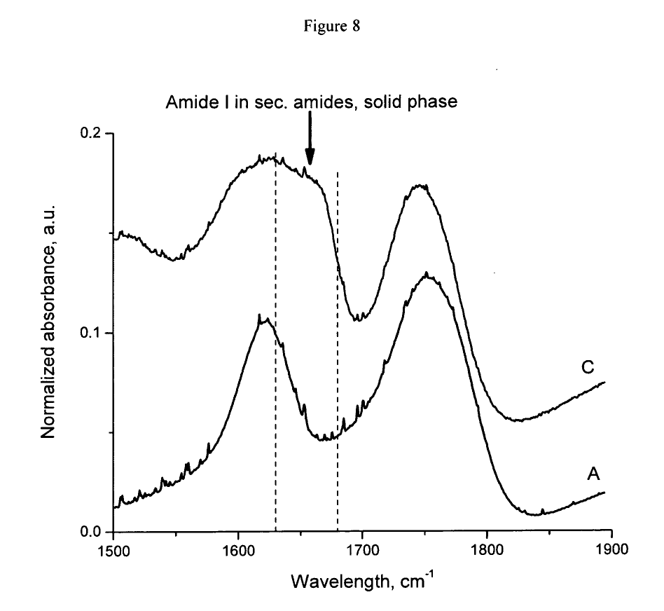

All these spectral features suggest the presence of aliphatic amine in C but do not confirm the fact of covalent linking. The decisive proof of covalent EDA attachment to the surface of nanodiamond would be an observation of amide bond formation upon conversion A into C according to the Scheme I. The area of a complex amide vibration (C═O stretch+N—H bend) is shown in FIG. 8. FIG. 8 illustrates FTIR spectra in amide bond vibrations area for initial purified ND A and aminated ND-EDA C.

Compared to spectrum of A a distinct shoulder is seen in spectrum of C in the range 1630-1680 cm⁻¹ corresponding to the so-called Amide I band (mainly C═O stretch in amide bond) of secondary amides. This evidence combined with above mentioned observations of increased N—H and C—H vibrations in C compared to A allows the conclusion that the material C is the result of conversion of A according to the Scheme 1. Though spectral evidences of amide bond formation are not very pronounced this agrees with the assumption of ˜20% carbon atoms situated at the surface in 5 nm spherical nanodiamond particles. Taking into consideration that i) not all of those 20% surface carbon atoms bear COOH groups, ii) not all of those COOH groups are enough reactive and iii) the yield of reactions in FIG. 6 is less than 100%, this small signal of amide bonds is not surprising and may correspond to the maximal possible degree of functionalization of nanodiamond.

### Example 3

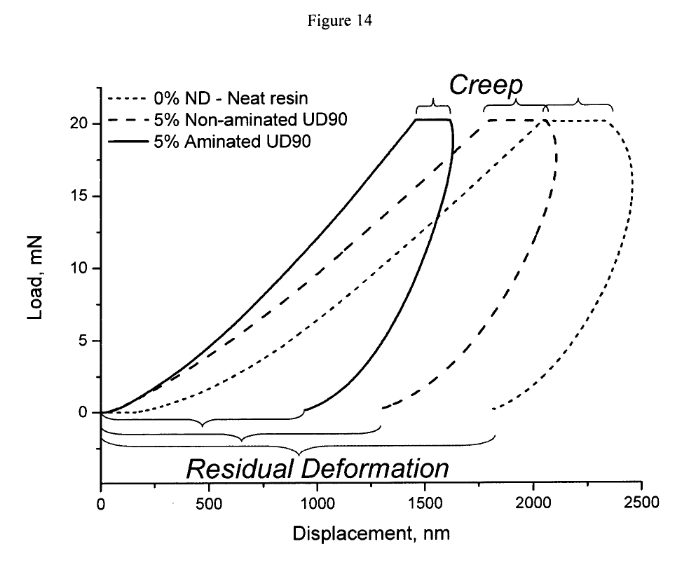

Composites of neat and aminated ND powders have been produced with x125A epoxy resin using x125B hardener (PSI, Inc. USA). The expected structure of the resulting ND-epoxy composite with covalent bonds between the ND and polymer is shown in FIG. 13. Mechanical tests of the produced ND-epoxy samples were performed using MTS Nanoindenter XP and a spherical diamond indenter of 13.5 micron radius (FIG. 14), and the following table:

| SAMPLE | Modulus, GPa | Hardness, GPa |
|---|---|---|
| Neat epoxy (0% wt. ND) | 2.7 | 0.17 |
| 5% wt. oxidized ND | 5 | 0.18 |
| 5% wt. aminated ND | 6.3 | 0.23 |

Nanoindentation shows an increase in Young's modulus, hardness and elastic recovery for the composites with aminated nanodiamond as compared to epoxies with neat or oxidized nanodiamond. Especially notable is the almost two-fold decrease in creep rate and significant increase in hardness, which were only observed for composites with aminated nanodiamond. These results provide an indication of covalent bonding of nanodiamond to the polymer matrix.

### Example 4

Epoxy-nanodiamond composites were manufactured by mixing nanodiamond with epoxy resin on a hotplate for one week to achieve an even dispersion and to allow modified nanodiamond to bond with the polymer chains. After mixing, a hardening agent was added to cross-link the polymer chains and polymerize the sample.

Cross-sectional specimens were cut, mounted and ground using 800 and 1200 grit silicon carbide paper and polished using alumina powders of decreasing size, 5, 1, 0.3, and 0.05 μm.

Samples were indented at constant strain rate for conversion to stress-strain curves, and at varying loads and varying loading rate. Scratches were also produced.

Nanoindentation tests were performed of cast epoxy mixed with nanodiamond and epoxy covalently bound to nanodiamond. Indentation was performed at constant strain for conversion to stress-strain curves, with the following results:

| | Modulus, | Hardness, | Creep | |
|---|---|---|---|---|
| Sample | GPa | GPa | rate, nm/s | Recovery, % |
| 0 wt % ND | 2.7 | 0.17 | 50 | 10 |
| 1 wt % | 2.9 ± 0.6 | 0.17 ± 0.01 | 50 | 16 |
| UD90 | | | | |
| 1 wt % | 3.7 ± 0.6 | 0.18 ± 0.01 | 40 | 5 |
| aminated | | | | |
| UD90 | | | | |
| 5 wt % | 5 ± 0.1 | 0.18 ± 0.01 | 60 | 17 |
| UD90 | | | | |
| 5 wt % | 6.3 ± 0.1 | 0.23 ± 0.01 | 30 | 31 |
| aminated | | | | |
| UD90 | | | | |

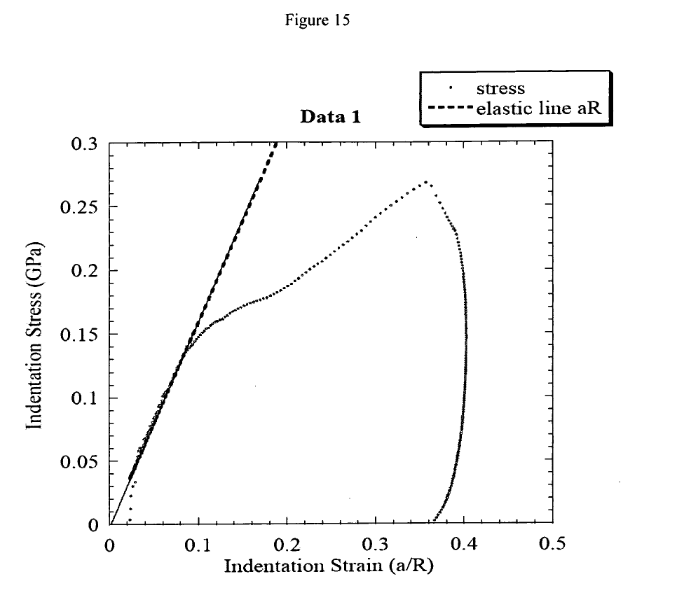

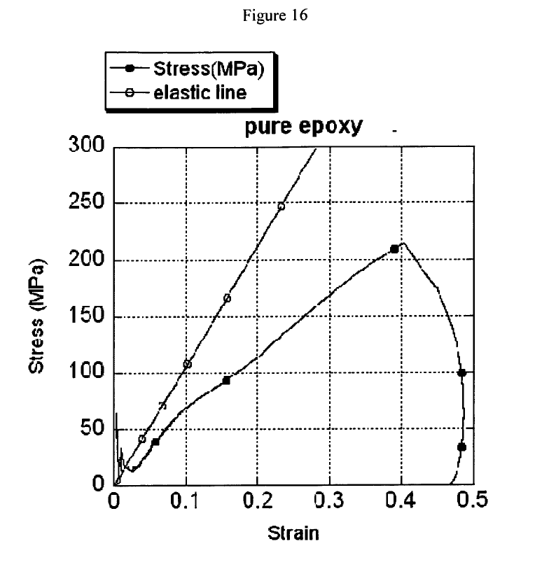

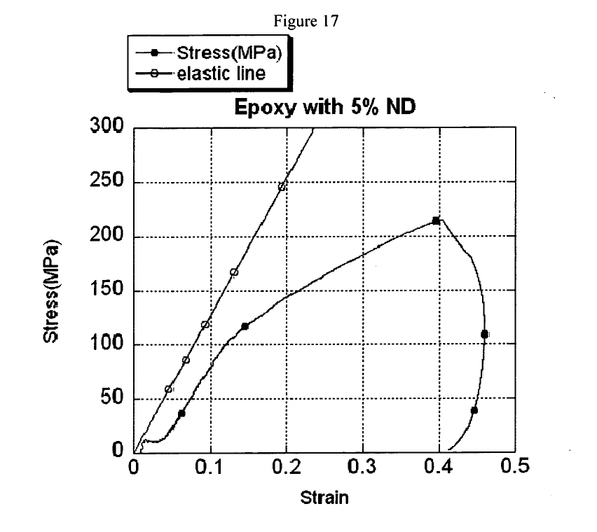

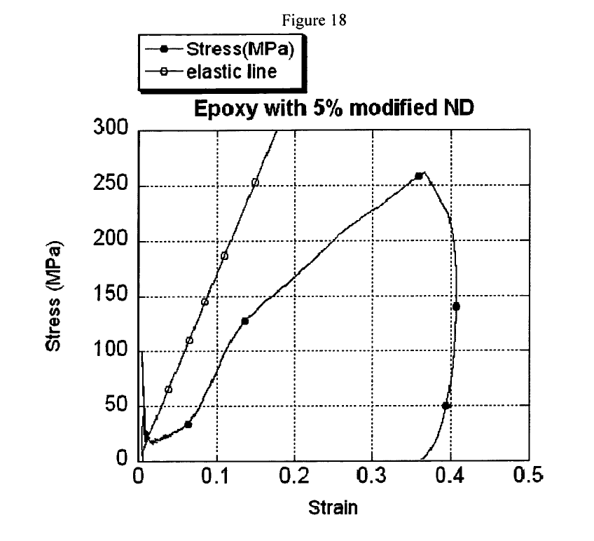

The nanoindentor was operated at the following conditions: the tip was a 13.5 micron spherical indenter; the load was 20 mN; the strain rate was 0.05/s. The results are further illustrated in FIG. 14 and FIG. 15. FIG. 15 illustrates a stress-strain curve for an epoxide polymer including 5 weight percent aminated nanodiamond. Further stress-strain curves are illustrated for pure epoxide polymer (FIG. 16, E=2.5 GPa, σy=70 MPa), epoxide polymer with 5 weight percent nanodiamond (FIG. 17, E=3 GPa, σy=120 MPa), and epoxide polymer with 5 weight percent aminated nanodiamond (FIG. 18, E=4 GPa, σy=130 MPa). The stress-strain curves in FIGS. 16-18 were derived from the arrays of indents performed at constant strain rate shown previously.

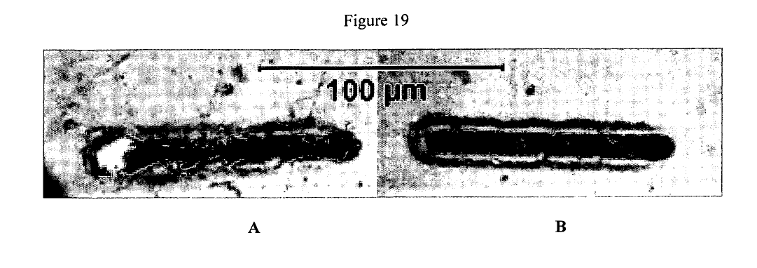

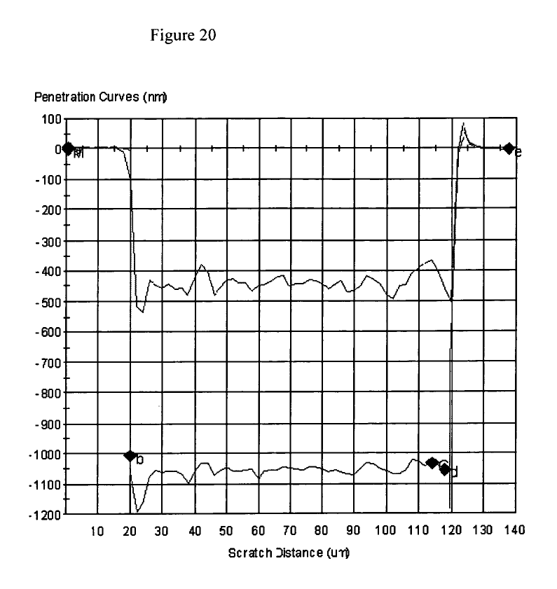

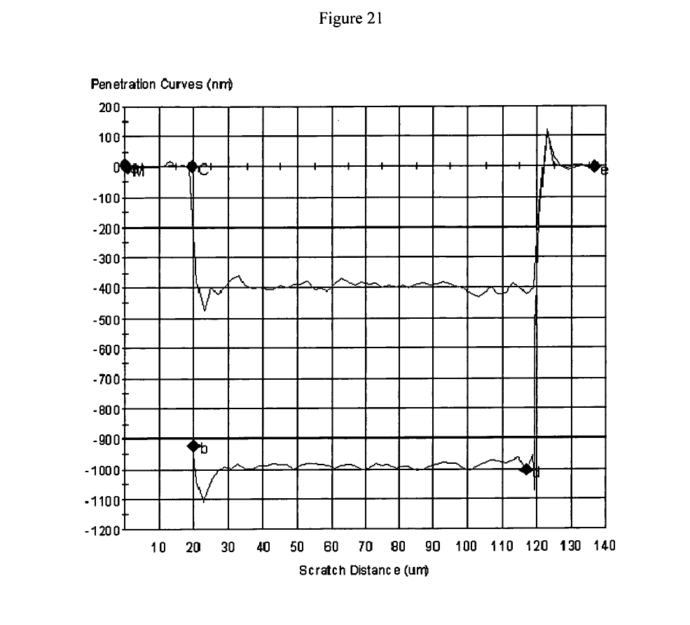

To composites including epoxide polymers and nanodiamonds, spherical scratches using tip of 1 μm radius were generated using a load of 20 mN for a length of 100 μm. The scratches are depicted visually in FIG. 19, for conventional epoxide polymer (A) and for composite of epoxide polymer and 5 weight percent aminated nanodiamonds (B). The nanodiamond composite showed improved scratch resistance, further illustrated by comparing the penetration curves for conventional epoxide polymer (FIG. 20) and for composite of epoxide polymer and 5 weight percent aminated nanodiamonds (FIG. 21). The conventional epoxide polymer experienced brittle cracking, whereas the composite of epoxide polymer and 5 weight percent aminated nanodiamonds experienced ductile pileup.

### Example 5

Nanodiamond powder (ND) (UD90 grade) was produced by detonation synthesis and supplied by Nanoblox, Inc. (Boca Raton, Fla.). The detonation soot was purified by the manufacturer using a multistage acidic treatment with nitric and sulfuric acids. Oxidized UD90 was then obtained through an oxidative purification in air at 425° C. to selectively remove non-diamond carbon phases. It was subsequently treated in 35 wt % HCl at 100° C. for 24 h to remove the metals and metal oxides by transforming them into water-soluble salts. In addition to purification, the HCl treatment of ND increases the number of surface carboxylic groups.

Semicrystalline Polyamide-11 (PA11) powder, available commercially as RILSAN D, “French Natural ES” (Arkema, Inc., King of Prussia, Pa.) with a 80 μm particle size (D80) and PAN bought from Scientific Polymer Products, were used.

PA11 was dissolved in a mixture of formic acid (FA) and dichloromethane (DCM) in a volume ratio of 1:1 at an 8 wt % concentration3. After the ND was dispersed in FA and DCM by sonication for 1 hour, it was mixed with the predissolved PA11 at a concentration of 2.5, 10, 20, 30 and 40 weight percent relative to the polymer. The use of the prolonged sonication helped to obtain well dispersed ND which, upon the mixing with the PA 11 solution is surrounded by polymer chains preventing reagglomeration of ND. Carboxyl groups at the surface of the ND are thought to interact with nitrogen atoms of the amide bonds in the backbone of PA11 chain electrostatically or through the formation of hydrogen bonds. Due to the stronger ND-polymer interactions, we expect a better dispersion and an improved polymer-filler interface.

The other polymer (PAN) was added to a dispersion of ND in dimethylformamide (DMF) at 6 weight percent polymer to solvent concentration to give 0-90 weight percent ND load relative to the polymer.

A Nanofiber Electrospinning Unit (NEU from Kato) was used to produce the nanofibers, which were electrospun at a voltage around 20 kV, with a syringe pump speed of 0.015 cm/min at a spinning distance of 15 cm in a horizontal syringe configuration.

The fibers were collected on aluminum foil or TEM grids. The samples for the UV/Vis spectroscopy were electrospun using aluminum foil wrapped glass slides (2 cm×2 cm) and then fused at ˜180° C. The thickness of fiber mats is controlled by the electrospinning time. Samples for nanoindentation were electrospun onto Si substrates. The fibers were then fused at 180° C. for 30 min to produce a thin polymer film4. The average thickness of the film as measured using optical profilometry was 2.6±0.4 μm. Nanoindentation and profilometry tests were performed on the films containing 0, 2.5, 10, and 20 weight percent of ND in PA11 since the samples with a higher content of ND had a less smooth surface, which may lead to large errors.

SEM analysis was performed using a Zeiss Supra 50 VP field emission SEM and a FEI XL30 environmental SEM (ESEM). The images with ESEM were taken with the SE detector, spot size 3 and an accelerating voltage of 3 kV. The images with the Zeiss were taken using an In Lens detector at ˜2 kV and a working distance of ˜4 mm in high vacuum mode. UV/Vis spectra were recorded using a Perkin Elmer, UV/Vis Lambda 35 spectrometer in a reflectance configuration. An optical profilometer Zygo New View 6000 was used to measure the thickness of the films. Depth sensing indentation and scratching was carried out at room temperature using Nanolndenter XP (MTS Corp.) equipped with a continuous stiffness measurements (CSM) attachment. The indents were performed with a spheroconical diamond tip of 13.5 μm radius. The indentation depth was 300 nm (˜10% of the coating thickness) to minimize substrate effects on the measured properties. The scratches were performed using a Berkovich tip and increasing the load linearly from 0 to 5 mN over a length of 500 nm.

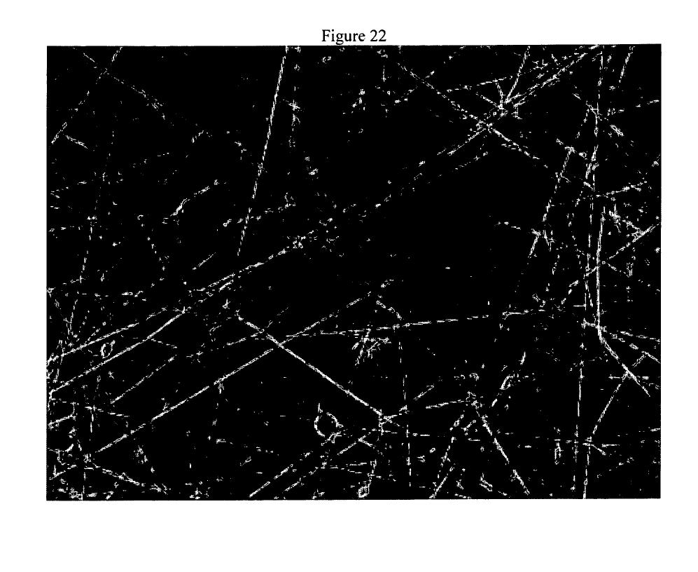

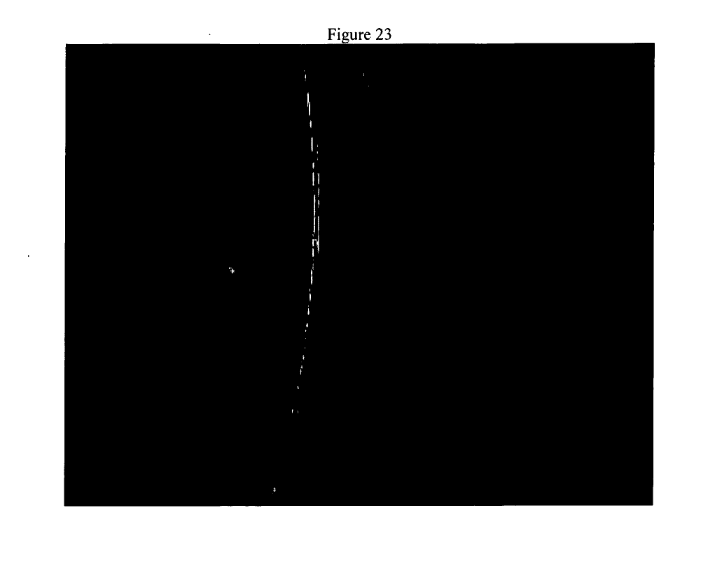

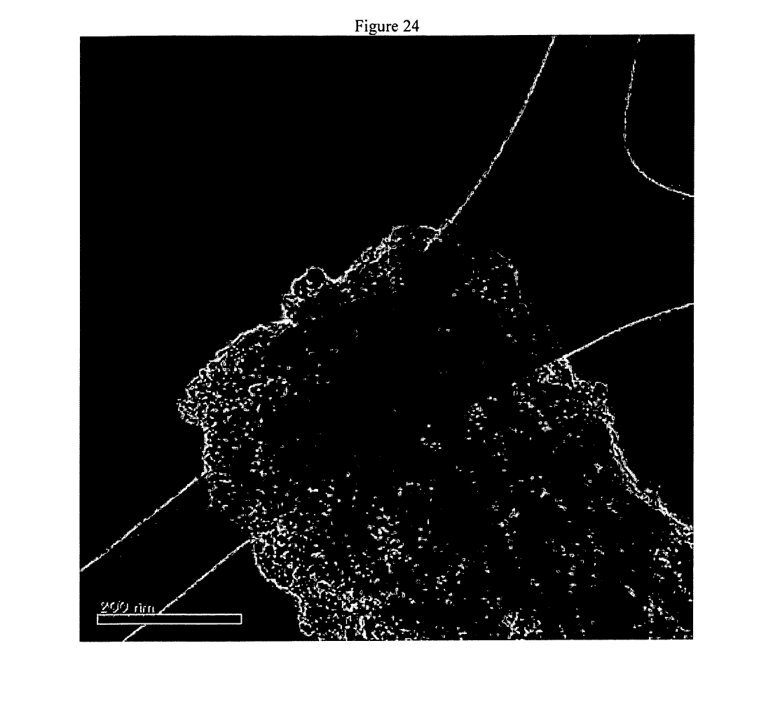

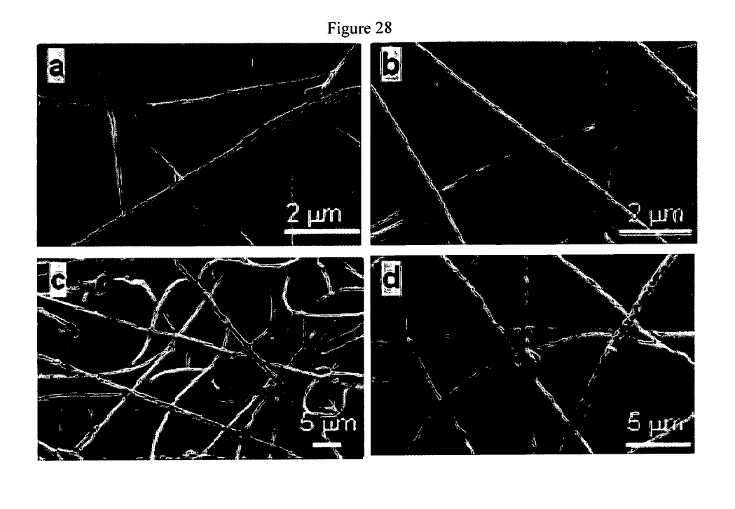

Nanofibers of PA11-ND were obtained at concentration of ND ranging from 0 to 40 wt %. Example nanofibers are depicted in FIG. 22, FIG. 23, and FIG. 24. As the diamond content inside the fibers increases, so does the surface roughness, as seen in FIG. 28. FIG. 28 depicts SEM micrographs of PA 11 electrospun fibers with varying loading of nanodiamond: (a) 2.5 wt %, (b) 10 wt %, (c) 20 wt % and (d) 40 wt %. A lower polymer concentration, relative to the solvent, decreases the viscosity of the solution, allowing a higher load of nanodiamonds in the fibers to be obtained. Nanofibers of PAN-ND with a 6 wt % of polymer relative to solvent, were obtained in the range of 0 to 60 wt % of ND.

For both polymers, it has been observed that a higher diamond content results in an increase of suspension viscosity, thus shifting electrospinning conditions away from optimal and leading to an irregular shape of the fibers and an increase in size and number of beads.

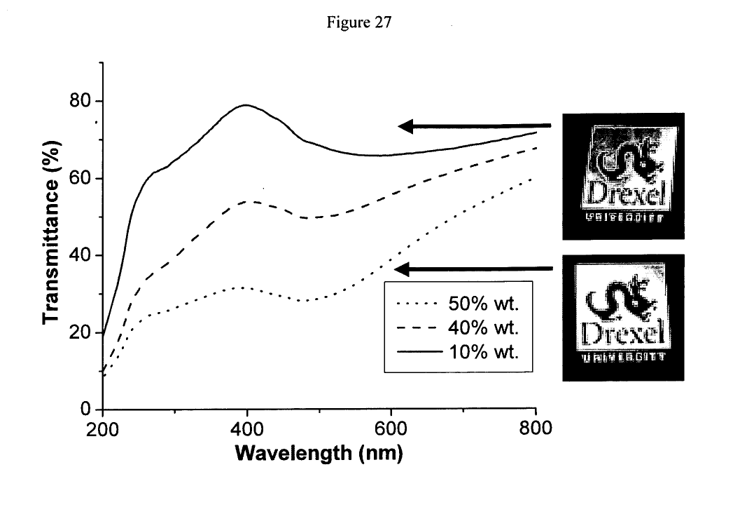

Heating of the electrospun polymer-ND nanofibers results in the formation of ND-polymer films with strong adhesion to the substrate. As-spun fiber mats look white or light grey depending on the amount of ND in them. This is due to light scattering by the nanosized fibers, gaps between which are comparable to the wavelength of visible light. Heating to the melting temperature of the polymer makes fiber mats transparent due to fibers fusing and spreading of the melt over the substrate. UV/Vis spectra of PAN-ND samples with different contents of ND were recorded against a film of pure fused PAN as a reference. As expected, film transmittance decreases as the ND content increases from 10 wt. % to 40 wt. % and finally to 50 wt. % (FIG. 27). FIG. 27 depicts UV/VIS spectra of PAN-ND films with different content of ND. Optical images of the films with 10 and 50 wt % are shown on the right. Nanodiamond is known to absorb in the deep UV and transmit in the visible and IR range 7. Some adsorption in the visible range may be due to about 5% of sp2 carbon present on the surface of oxidized ND2. Comparison of transmittance values at 500 nm shows that, although there is an increase in the V is absorption with the ND load, the sample at 40 wt. % of ND still retains 50% of transmittance. While fairly high optical transparency can be maintained up to 40 wt % ND, all samples intensively absorb in the deep UV, showing that even at small additions of nanodiamonds these films may be used as UV protective coatings.

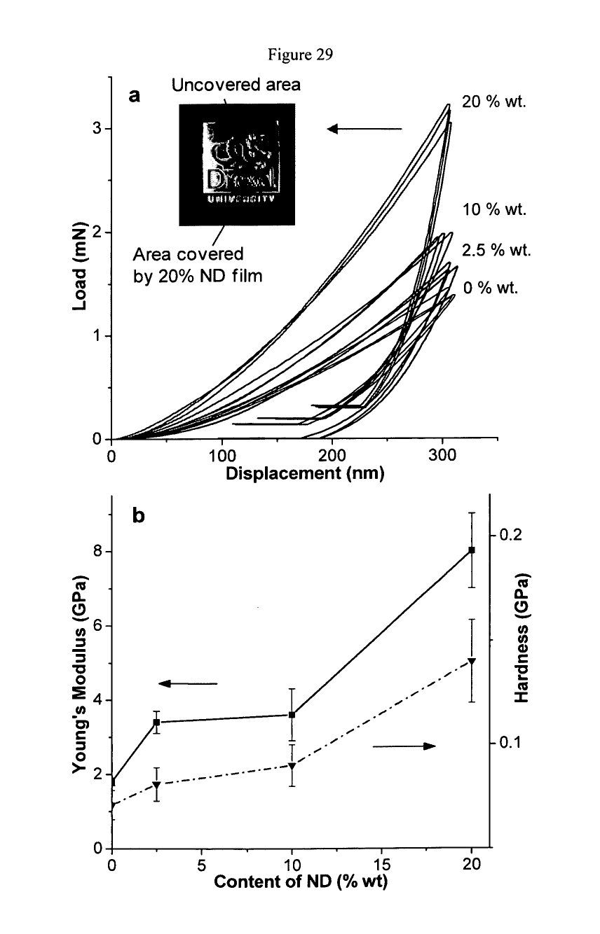

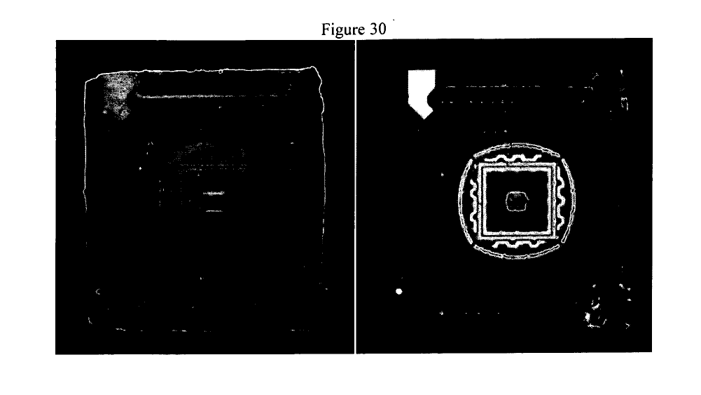

PA11-ND films were obtained on glass, steel and silicon with different loadings of ND. Experimental load-displacement curves for the films on silicon with 0 to 20 wt % of ND are shown in FIG. 29. FIG. 29 depicts load—displacement curves (a) and hardness and Young's modulus (b) of PA 11-ND films with different content of nanodiamond. Inset in panel (a) shows a PA 11-ND film with 20 wt % of nanodiamond. It can be seen from the curves that whereas a load of 1.3 mN is enough to penetrate the pure PA 11 coating to the depth of 300 nm (FIG. 29a), a 3-times higher load is required to reach the same depth for a coating with 20 wt % ND. Hardness and Young's modulus values were calculated over the loading part from the CSM data as the averages of 10 different tests on the same sample (FIG. 29b). The nanocomposite with 20 wt % of ND demonstrated nearly four times higher Young's modulus and doubled hardness value as compared to the pure polymer. Scratch tests also show improvement in the hardness. Whereas the scratch generated by a sharp Berkovich indenter under 5 mN load in pure PA film was 1.7 μm deep (data not shown), the addition of just 10 wt % of ND decreased the scratch depth down to 0.8 μm.

### Example 6

The nanodiamond powder was produced via a detonation synthesis and supplied by Nanoblox, Inc. (USA). As-received nanodiamond powder (UD90 grade) has been thoroughly characterized using Raman spectroscopy, TEM, XANES, and FTIR. Before incorporation into the polymer matrix, UD90 has been purified by air oxidation to remove non-diamond carbon and further treated in concentrated HCl at 100° C. for 24 h to remove the metals and metal oxides by transforming them into water-soluble salts. Finally the ND powder was rinsed with DI water until neutral pH. In addition to purification, HCl treatment of ND increases the number of surface carboxylic groups thus resulting in better suspension stability due to increased negative charge on the surface of the particles upon dissociation of —COOH. Purified ND was dispersed in a solvent that was compatible with the particular polymer that is to be dissolved and then electrospun.

Two similar methods of incorporation were employed based on the matrix polymer. First, HCl treated nanodiamond was added to dimethylformamide (DMF) under stirring, sonication and shaking. The PAN powder (purchased from Scientific Polymer Products) was then added to the suspension to produce a 6% by weight polymer solution with varying amounts of nanodiamond ranging from 0 to 90% by weight of the produced fiber. For the second polymer, PA 11 (D80 powder provided by Arkema, Inc.), the nanodiamond was dispersed in a solvent composed of formic acid (FA) and dichloromethane (DCM) in a 1:1 volume ratio. This same solvent has been previously used for electrospinning PA 11 in the concentration ranges of 1-10 wt. %.15, 24 After the ND was dispersed in FA and DCM at concentrations 2.5, 10, 20, 30 and 40% by weight of the produced fiber, the dispersions were sonicated for 1 hour, and then the predissolved PA 11 was added to them and thoroughly mixed.

The electrospinning machine, a Kato Nanofiber Electrospinning Unit (NEU), was supplied by NanoBlox, Inc. The electrospinning has been performed using a horizontal and slightly inverted setup (between 0 and 45°). The unit was operated at ˜23 kV for the PAN solution and between 15 and 20 kV for the PA 11 solution. A spinning distance between the syringe needle tip and the collection plate was maintained at 15 cm and the dispersions were pumped through the syringe at 0.015 cm/min in both cases. The fibers were collected on aluminum foil or TEM grids for subsequent microscopy studies.

For UV/Vis spectroscopy studies, samples of 10 wt. %, 40 wt. % and 50 wt. % ND in PAN were electrospun onto aluminum foil covered glass slides (2 cm×2 cm) which were rotated (180°) at the mid-point of the 20 minutes spinning time. The thickness of the produced fiber mats is controlled by the electrospinning time: an increased time deposits thicker fiber mats. Samples were heated to 200° C. and also left as spun for comparison purposes. Samples for nanoindentation were electrospun onto silicon substrates (. The PA 11 fibers were then fused at 180° C. for 30 min. Sample from each set was scratched with a razor blade in order to measure the film thickness using optical profilometry. The average thickness of the films spun for 20 min was 2.6±0.4 μm. The nanoindentation and scratches were performed on the films containing 0, 2.5, 10, and 20 wt. % of ND in PA 11 since the ones with a higher content of ND showed a less smooth surface, which may lead to large errors in the tests.

SEM analysis was performed using a Zeiss Supra 50 VP field emission SEM and a FEI XL30 environmental SEM (ESEM). The images were taken at ˜2 kV and a working distance of ˜4 mm in high vacuum mode. TEM was performed at the University of Pennsylvania, using a JEOL 2010F operated at 100 or 200 kV. UV/Vis spectroscopy was performed using a Perkin Elmer UV/Vis Lambda 35 spectrometer in reflectance mode. An optical profilometer Zygo New View 6000 was used to measure the thickness of the films. Depth sensing indentation and scratching was conducted at room temperature using Nanolndenter XP (MTS Corp.) equipped with a continuous stiffness measurements (CSM) attachment. The indents were performed with a spheroconical diamond indenter of 13.5 μm radius. The indentation was carried out to the depth of 300 nm (only ˜10% of the coating thickness) to minimize substrate effects on the measured properties. The scratches were performed using a Berkovich tip, increasing the load linearly from 0 to 5 mN over a length of 500 μm.

PAN-ND composites with a nanodiamond content in the range of 0-90 80 wt. % were obtained in the form of electrospun nanofiber mats and then studied using Scanning Electron Microscopy (SEM), Transmission Electron Microscopy (TEM) and UV/Vis spectroscopy. The fibers were also applied as a coating and tested for their UV/Vis absorbance for which they were melted to produce an optically transparent film. PA 11-ND composite nanofibers with up to 40% ND were electrospun into mats and characterized using SEM to show the applicability of the concept as it is used in other polymer systems. PA 11 fibers were further investigated as wear-resistant coating on various substrates. Glass, steel, silicon and aluminum were used as substrates and transparent coatings with excellent adhesion (no delamination) have been manufactured on all of the above surfaces.

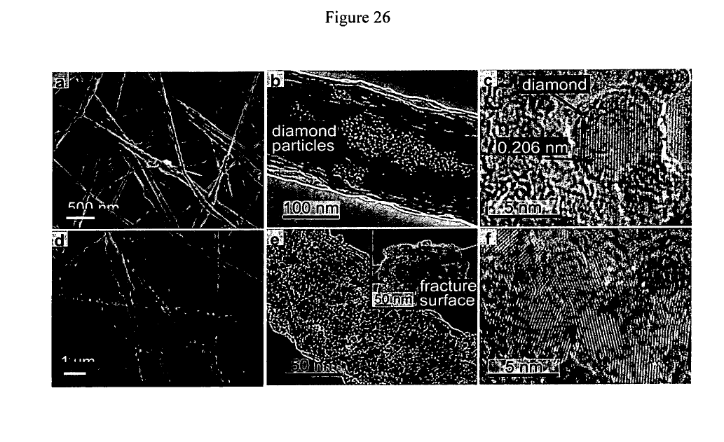

Representative SEM and TEM images of the ND-PAN fibers are shown in FIG. 26. Pure PAN nanofibers (not shown) were thin (34 nm average diameter) and smooth with a few instabilities per fiber, perhaps due to not quite uniform and stable electrospinning conditions. Lower polymer concentration, relative to the solvent, decreases the viscosity and increases the highest obtainable loading of nanodiamond. 10 wt. % ND-PAN fibers (FIGS. 26a, b) show a non-uniform distribution and some agglomeration of ND in the matrix, but still they are smooth and in this respect similar to the pure PAN fibers. The average fiber diameter decreases with the increasing diamond content from 0 to 17 wt. % reaching about 15 nm. Further increases of ND content lead to larger fibers with less uniform diameters. The size and number of beads increases as the concentration of nanodiamond increases between 20 and 60 wt. %. Although it is still possible to produce fibers of 60% ND-PAN composite (FIG. 26d) their diameter is larger and less uniform. However, while fiber diameter distribution broadens as the concentration of the ND increases above 20 wt. %, the fibers appear to have a more uniform distribution of diamond particles in the polymer (compare FIGS. 26b and 26e).

It is particularly important to notice a uniform distribution of nanodiamond throughout the fiber even at high ND loads, as seen in FIG. 26e. Fast evaporation of the solvent during electrospinning suppresses reagglomeration resulting in significantly improved nanodiamond dispersion, compared to traditional mixing techniques, and in an increased area of contact between the polymer and the nanodiamond particles. These fibers fail in a brittle manner at high diamond loads (inset in FIG. 26e), with no necking or crazing, that were observed for pure PAN or CNT-containing PAN nanofibers. Diamond crystals were not affected by the spinning process as shown in FIGS. 26c, f. With an increase of the diamond content above 60 wt. %, the viscosity of ND-PAN suspension increases, thus making electrospinning conditions non-optimal. However, even at high concentration it is still possible to produce beaded nanofibers. It gives an opportunity to obtain a polymer-bonded electrosprayed ND coating on virtually any surface.

PAN fiber mats spun for 10 and 20 minutes were white or light grey and translucent, independent of the diamond content. This is due to light scattering on nanofibers, gaps between which (FIG. 26a, c) are comparable to the wavelength of visible light. Heating to 200° C. makes fiber mats transparent due to fibers fusing to the surface and material uniformly spreading over the surface (the softening temperature of PAN is ˜180° C.), leading to the formation of a continuous film, as seen in FIG. 27.

PAN electrospun fiber mats with different concentrations of ND were heated for 20 minutes at 200° C. and a film of pure fused PAN was used as a reference for the ND-PAN samples in UV/Vis measurements. All films used for UV/Vis measurements had approximately the same thickness of 2.6±0.4 nm. As can be seen in FIG. 27, the transmission decreases as the ND content increases from 10 wt. % to 40 wt. % and finally to 50 wt. %. Nanodiamond is known to absorb in deep UV and transmit in the visible and IR range. Some adsorption in the visible range may be due to about 5% of sp2 carbon present on the surface of oxidized ND. There is a decrease of about 25% in the transmission from 10 to 40 wt. % and a further decrease of ˜25% from the 40 to 50 wt. % samples.

### Example 7

Electrospinning methods have also been applied to PA 11 fibers. Similar to the PAN fibers, fused films of electrospun PA 11 fibers with ND incorporation show absorption in the UV range, but remain optically transparent up to at least 40 wt % of diamond (FIG. 31, optical images of electrospun PA 11 nanofibers with 10 wt. % ND on a computer chip before (left) and after (right) heating that leads to a formation of a transparent film). The ability to absorb UV radiation while remaining transparent in the visible range is advantageous for many applications, such as glass coating and protective layers on UV sensitive materials. Similar to the PAN fibers, as the diamond content increases so does the surface roughness of the PA 11 fibers, as seen in FIG. 28, indicating SEM images of PA 11 electrospun fibers with varying loading of nanodiamond: (a) 2.5 wt. %, (b) 10 wt. %, (c) 20 wt. % and (d) 40 wt. %. The appearance of the fibers changes from a fibrous to fibers with a rougher surface to droplets as the concentration of diamond changes. This is partially dependant on the increasing the viscosity of the solution due to an increase in the concentration of nanodiamond. However, addition of nanodiamond, even up to 40 wt. %, does not increase viscosity as much as just a few weight percent of nanotubes, thus making nanodiamond an more attractive filler material for high loading of polymer matrix nanofiber composites. Electrospun PA 11 fibers have a larger diameter than PAN fibers and non circular shape. The low conductivity of PA11 was not affected by the addition of ND (not shown), enabling its use as an insulating and protective coating on electronic devices (FIG. 31).

Addition of nanodiamond has significantly improved mechanical properties of PA 11 films, raising their Young's modulus by a factor of 4 and more than doubling the hardness. It can be seen from FIG. 29 that if a load of 1.3 mN is required to penetrate the pure PA 11 coating to the depth of 300 nm (FIG. 29a), a 3-times higher load is required to reach the same depth for a coating with 20 wt % ND. If the scratch generated by a sharp Berkovich indenter under 5 mN load in pure PA was 1.7 μm deep, addition of merely 10 wt % of ND led to a 0.8 μm deep scratch. Thus, scratch tests have confirmed improved hardness and scratch resistance of PA 11 after adding ND.

The quality of fibers is further improved by optimization of electrospinning process via incorporation of a conducting salt or further improvement of the diamond dispersion. According to TEM, at 50-60 wt. % of ND, the density of the nanodiamonds inside the fibers is so high that the fibers may be well considered as ND fibers with a polymer binder.

### Example 8

To synthesize a nanodiamond-poly(epoxide) composite, 0.05 g of aminated UD90 powder was mixed with 10 ml of tetrahydrofuran in a 20 ml glass vial and sonicated in an ultrasound bath until there remained no visible pieces of the solid. 5 g of an epoxy resin x125A (PSI, Inc. USA) was dissolved in 10 ml of tetrahydrofuran and added into the vial with UD90 suspension, thus the total volume of liquid in the vial was brought to 20 ml. After that, a Teflon magnetic stirrer bar was added to the vial with UD90 suspension in the epoxy resin in THF solution, the vial was capped and left under continuous stirring on a stirring hotplate at 70° C. for a week to accomplish the reaction between the epoxy and aminoterminated UD90. Then, the cap was open and the vial was left under continuous heating and stirring for 24 h more to evaporate THF. After the solvent removal, 1.5 g of x125B curing agent (PSI, Inc. USA) was added and carefully mixed with the content of the vial. To cure the UD90-epoxy composite, the content of the vial was cast into an aluminum mold and put into a stove at 170° C. for 24 h. The resulting solid UD90-epoxy composite with content of UD90 1% wt. was removed from the mold and subjected to further tests.

### Example 9

To synthesize a nanodiamond-poly(epoxide) composite, 0.5 g of aminated UD90 powder was mixed with 50 g of Epon 825 Bisphenol A epoxy resin in Thinky planetary centrifugal mixer for 3 min. Then 12.5 g of the EPI-CURE liquid aliphatic amine curing agent (Epon) was added and the mixture was mixed in the Thinky mixer for another 3 min. To cure the UD90-epoxy composite, it was cast into an aluminum mold and held in a stove at 150° C. for 3 h to accomplish the reaction between the aminogroups of UD90 and epoxy resin. Then the temperature was risen to 170° C. and the composite was left in the stove at this temperature for 5 h more. The resulting solid plaque of UD90-epoxy composite with content of UD90 1% wt. was removed from the mold, cut, milled and subjected to further tests.

What is claimed:

1. A composition, comprising:
at least one functionalized nanodiamond, comprising at least one acyl group linked to one or more surface groups.

2. The composition of claim 1, wherein the one or more surface groups comprise a hydrocarbon chain, an alkene, an alkyne, a monomer, an aromatic molecule, a nucleophile, a fluorescent species, an antibody, a ligand, an amine, an amino group, a thiol, a sulfur, an acid, a base, an alcohol, a monomer, a polymer, a metal, a ceramic, a protein, a nucleic acid, a biochemical, or any combination thereof.

3. The composition of claim 1, wherein the at least one functionalized nanodiamond comprises a characteristic cross-sectional dimension in the range of from about 1 nm to about 50 nm.

4. The composition of claim 1, wherein the at least one functionalized nanodiamond comprises a characteristic cross-sectional dimension in the range of from about 5 nm to about 20 nm.

5. The composition of claim 1, further comprising a solvent.

6. The composition of claim 5, wherein the at least one functionalized nanodiamond is dispersed in the solvent.

7. The composition of claim 5, wherein the solvent comprises toluene, benzene, dichloromethane, n-n dimethylformamide, acetone, ethanol, or any combination thereof.

8. A method for synthesizing a functionalized nanodiamond having at least one functionality, comprising:
reacting at least one nanodiamond with at least one donor species to give rise to at least one nanodiamond intermediate, the nanodiamond comprising a carboxylic acid group, an ester, or any combination thereof,
the nanodiamond intermediate comprising at least one acyl-halogen group or at least one acyl-tosylate group, or both;
reacting the at least one nanodiamond intermediate with at least one displacer group,
the displacer group displacing either an acyl-bound halogen or a tosylate, or both, of the at least one nanodiamond intermediate with at least one functionality, so as to give rise to at least one functionalized nanodiamond.

9. The method of claim 8, wherein the at least one nanodiamond is produced by detonation synthesis.

10. The method of claim 8, wherein the at least one donor species comprises a halogen-donating species or a tosylate-donating species.

11. The method of claim 10, wherein the at least one donor species reacts with a carboxylic acid group, an ester group, or both, of the at least one nanodiamond to give rise to an acyl-halogen group, an acyl-tosylate group, or both.

12. The method of claim 10, wherein the halogen-donating species comprises SOX₂, PX₃ or any combination thereof, wherein X is a halogen.

13. The method of claim 8, wherein the displacer group comprises an amino group, an alcohol, an amide, an aminoacid, a peptide, a hydroxyl group, a peptide, a protein, or any combination thereof.

14. The method of claim 8, wherein the at least one functionality comprises a hydrocarbon chain, an aromatic group, a nucleophile, a fluorescent species, an amino group, a thiol, a sulfur, an acid, a base, a ligand, an antibody, a hydroxyl group, a protein, a biological molecule, a monomer, a nucleic acid, a polymer, a metal, an alcohol, or any combination thereof.

15. The method of claim 8, wherein the at least one nanodiamond comprises a characteristic cross-sectional dimension in the range of from about 1 to about 10 nm.

16. The method of claim 15, wherein two or more nanodiamonds are agglomerated into one or more particles having a characteristic cross-sectional dimension in the range of from about 20 nm to about 500 nm.

17. The method of claim 15, wherein two or more nanodiamonds are agglomerated into one or more particles having a characteristic cross-sectional dimension in the range of from about 100 nm to about 200 nm.

18. The method of claim 8, wherein the at least one functionalized nanodiamond comprises a characteristic cross-sectional dimension in the range of from about 1 nm to about 50 nm.

19. The method of claim 8, wherein the at least one functionalized nanodiamond comprises a characteristic cross-sectional dimension in the range of from about 10 nm to about 40 nm.

20. The method of claim 8, wherein two or more functionalized nanodiamonds are agglomerated into particles having a characteristic cross-sectional dimension in the range of from about 20 nm to about 500 nm.

21. A functionalized nanodiamond made according to the method of claim 8.

22. A nanodiamond tracer, comprising:
a functionalized nanodiamond comprising at least one functionality linked to a nanodiamond by an acyl linkage,
the functionalized nanodiamond being capable of emitting a signal.

23. The nanodiamond tracer of claim 22, wherein the at least one functionality comprises one or more hydrocarbon chains, aromatic groups, nucleophiles, fluorescent species, amino groups, thiols, sulfurs, acids, bases, proteins, biological molecules, monomers, polymers, metals, alcohols, radioactive species, magnetic species, or any combination thereof.

24. The nanodiamond tracer of claim 22, wherein the signal comprises a visual signal, an infrared signal, an ultraviolet signal, a radioactive signal, a magnetic signal, an electrical signal, an electromagnetic signal, or any combination thereof.

25. The nanodiamond tracer of claim 24, wherein the functionalized nanodiamond is capable of emitting one or more signals under illumination.

26. The nanodiamond tracer of claim 25, wherein the illumination comprises visible light, ultraviolet light, infrared light, x-rays, gamma rays, electromagnetic radiation, radio waves, radioactive particles, or any combination thereof.

27. The nanodiamond tracer of claim 22, wherein the functionalized nanodiamond comprises a characteristic cross-sectional dimension in the range of from about 1 nm to about 50 nm.

28. The nanodiamond tracer of claim 22, wherein the functionalized nanodiamond comprises a characteristic cross-sectional dimension in the range of from about 10 nm to about 30 nm.

29. The nanodiamond tracer of claim 22, wherein two or more functionalized nanodiamonds are agglomerated into particles having a characteristic cross-sectional dimension in the range of from about 20 nm to about 500 nm.

30. A method for sensing a nanodiamond, comprising:
detecting at least one signal emitted by at least one nanodiamond comprising at least one acyl linkage covalently bonded to at least one functionality capable of generating a signal.

31. The method of claim 30, wherein the at least one functionality comprises a hydrocarbon chain, an aromatic, a fluorescent dye, a fluorescent protein, a heterocyclic compound, a magnetic molecule, a radioactive species, or any combination thereof.

32. The method of claim 30, wherein the nanodiamond is illuminated with visible light, ultraviolet light, infrared light, an x-ray, a gamma ray, a radioactive particle, an electromagnetic wave, an electric field, or any combination thereof.

33. The method of claim 30, wherein the nanodiamond has a characteristic cross-sectional dimension in the range of from about 1 nm to about 50 nm.

34. The method of claim 30, wherein the nanodiamond has a characteristic cross-sectional dimension in the range of from about 10 nm to about 30 nm.

35. The method of claim 30, wherein two or more nanodiamonds are agglomerated into particles having a characteristic cross-sectional dimension in the range of from about 20 nm to about 500 nm.

36. The method of claim 30, wherein the at least one signal comprises a visual signal, an infrared signal, an ultraviolet signal, a radioactive signal, a magnetic signal, or any combination thereof.

37. The method of claim 30, wherein the signal is detected using any of the following methods: visually inspecting, monitoring electromagnetic radiation, monitoring radioactive emissions, monitoring a magnetic signal, or any combination thereof.

38. An detection system, comprising:
at least one functionalized nanodiamond, the functionalized nanodiamond comprising at least one acyl linkage covalently bonded to at least one functionality capable of generating a signal; and
a detector capable of detecting one or more signals generated by the nanodiamond.

39. The detection system of claim 38, wherein the at least one functionality capable of generating a signal comprises a hydrocarbon chain, an aromatic, a fluorescent dye, a fluorescent protein, a heterocyclic compound, a magnetic molecule, a ligand, an antibody, a radioactive species, or any combination thereof.

40. The detection system of claim 38, further comprising an excitation source.

41. The detection system of claim 38, wherein the excitation source emits visible light, ultraviolet radiation, x-rays, magnetic waves, infrared light, microwaves, radio waves, or any combination thereof.

42. The detection system of claim 40 wherein the excitation source is capable of eliciting one or more signals from the at least one functionalized nanodiamond.

43. The detection system of claim 38, wherein the functionality of the nanodiamond is characterized as being capable of any of the following: preferentially binding to one or more specific cells of an organism, preferentially binding to one or more specific materials, preferentially binding to one or more molecules of an organism, or any combination thereof.

44. The detection system of claim 38, wherein the at least one nanodiamond has a characteristic cross-sectional dimension in the range of from about 1 nm to about 50 nm.

45. The detection system of claim 38, wherein the at least one nanodiamond has a characteristic cross-sectional dimension in the range of from about 10 nm to about 30 nm.

46. The detection system of claim 38, wherein two or more nanodiamonds are agglomerated into particles having a characteristic cross-sectional dimension in the range of from about 20 nm to about 500 nm.

47. A method for synthesizing a polymer-nanodiamond composite, comprising:
reacting at least one nanodiamond with at least one donor species to give rise to at least one nanodiamond intermediate, the nanodiamond comprising a carboxylic acid group, an ester, or any combination thereof,
the nanodiamond intermediate comprising at least one acyl-halogen group, at least one acyl-tosylate group, or both;
reacting the at least one nanodiamond intermediate with at least one displacer group,
the displacer group displacing an acyl-bound halogen, an acyl-bound tosylate, or both, of the nanodiamond intermediate with at least one first monomer, so as to give rise to at least one monomer-bearing nanodiamond; and
polymerizing at least one monomer-functionalized nanodiamond with a second monomer to give rise to a polymer-nanodiamond composite.

48. The method of claim 47, wherein the donor species comprises a halogen-donating species or a tosylate-donating species.

49. The method of claim 48, wherein the at least one donor species converts a carboxylic acid group, an ester group, or both, of the at least one nanodiamond to an acyl-halogen group, an acyl-tosylate group, or both.

50. The method of claim 49, wherein the halogen-donating species comprises SOX₂, PX₃ or any combination thereof, wherein X is a halogen.

51. The method of claim 47, wherein either the monomer-linked nanodiamond, the second monomer, or both, comprises an alkene, an alkyne, a styrene, an amide, an alcohol, an amino acid, an ester, or any combination thereof.

52. The method of claim 47, wherein the displacer group comprises lithium, magnesium, an amide, an hydroxyl, a hydride, or any combination thereof.

53. The method of claim 47, wherein the monomer-linked nanodiamond and the second monomer have the same composition.

54. The method of claim 47, wherein the monomer-linked nanodiamond and the second monomer have different compositions.

55. The method of claim 47, wherein the nanodiamond has a characteristic cross-sectional dimension in the range of from about 1 nm to about 50 nm.

56. The method of claim 47, wherein the nanodiamond has a characteristic cross-sectional dimension in the range of from about 10 nm to about 30 nm.

57. The method of claim 47, wherein two or more nanodiamonds are agglomerated into particles having a characteristic cross-sectional dimension in the range of from about 20 nm to about 500 nm.

58. A polymer-nanodiamond composite made according to the method of claim 47.

59. A composition of matter comprising at least one nanodiamond covalently bonded to a polymer.

60. The composition of claim 59, wherein the polymer comprises two or more monomer subunit species, and at least one of the monomer subunit species is covalently bonded to the at least one nanodiamond.

61. The composition of claim 59, wherein the at least one polymer comprises at least one of a styrenic polymer, an acrylic polymer, a fluoropolymer, a poly(epoxide), a poly(amide), a poly(ester), a poly(methylmethacrylate), a poly(ethylene), a poly(acrylonitrile), a poly(propylene), a thermoplastic poly(urethane), a poly(imide), a alkylene tetrafluoroethylene, a polycarbonate, a poly(ethylene oxide), a poly(caprolactone), or any copolymer or combination thereof.

62. The composition of claim 60, wherein at least one monomer subunit species comprises at least one of an amine, an alkene, an alkyne, a styrene, an amide, an alcohol, an amino acid, an ester, or any combination thereof.

63. The composition of claim 60, wherein at least one monomer subunit species comprises at least one amine.

64. The composition of claim 63, wherein the monomer subunit species comprising at least one amine is the only one of the monomer subunit species that is covalently bonded to the at least one nanodiamond.

65. The composition of claim 59, wherein the at least one nanodiamond is characterized as having a characteristic cross-sectional dimension in the range of from about 1 nm to about 50 nm.

66. The composition of claim 59, wherein the at least one nanodiamond is characterized as having a characteristic cross-sectional dimension in the range of from about 5 nm to about 20 nm.

67. The composition of claim 59, wherein the weight percent of nanodiamonds in the composition is in the range from about 0.01 percent to about 90 percent based on total weight of the composition.

68. The composition of claim 59, wherein the weight percent of nanodiamonds in the composition is in the range from about 0.01 percent to about 25 percent based on total weight of the composition.

69. The composition of claim 59, wherein the weight percent of nanodiamonds in the composition is in the range from about 0.5 percent to about 1 percent based on total weight of the composition.

70. The composition of claim 59, wherein the weight percent of nanodiamonds in the composition is in the range from about 0.01 percent to about 5 percent based on total weight of the composition.

71. The composition of claim 59, wherein the at least one nanodiamond is produced by detonation synthesis.

72. The method of claim 47, wherein the first monomer comprises an amine.

73. The method of claim 47, wherein the second monomer comprises an epoxide.

74. The method of claim 47, wherein the polymerizing gives rise to a poly(epoxide)-nanodiamond composite.

75. An article of manufacture comprising an electrospun fiber comprising a polymer and at least one nanodiamond.

76. The article of claim 75, wherein the polymer comprises at least one of a styrenic polymer, an acrylic polymer, a fluoropolymer, a poly(epoxide), a poly(amide), a poly(ester), a poly(methylmethacrylate), a poly(ethylene), a poly(acrylonitrile), a poly(propylene), a thermoplastic poly(urethane), a poly(imide), a alkylene tetrafluoroethylene, a polycarbonate, a poly(ethylene oxide), a poly(caprolactone), or any copolymer or combination thereof.

77. The article of claim 75, wherein the at least one nanodiamond is characterized as having a characteristic cross-sectional dimension in the range of from about 1 nm to about 50 nm.

78. The article of claim 75, wherein the at least one nanodiamond is characterized as having a characteristic cross-sectional dimension in the range of from about 5 nm to about 20 nm.

79. The article of claim 75, wherein the weight percent of nanodiamonds in the electrospun fiber is in the range from about 0.01 percent to about 90 percent based on total weight of the electrospun fiber.

80. The article of claim 75, wherein the weight percent of nanodiamonds in the electrospun fiber is in the range from about 10 percent to about 60 percent based on total weight of the electrospun fiber.

81. The article of claim 75, wherein the weight percent of nanodiamonds in the electrospun fiber is in the range from about 15 percent to about 30 percent based on total weight of the electrospun fiber

82. The article of claim 75, wherein the at least one nanodiamond is produced by detonation synthesis.

83. The article of claim 75, wherein the at least one nanodiamond is a functionalized nanodiamond.

84. The article of claim 75, wherein the at least one nanodiamond is covalently bonded to the polymer.

85. A process comprising electrospinning a mixture comprising a polymer and at least one nanodiamond.

86. The process of claim 85, further comprising sonicating the mixture of polymer and at least one nanodiamond.

87. The process of claim 85, wherein the polymer comprises at least one of a styrenic polymer, an acrylic polymer, a fluoropolymer, a poly(epoxide), a poly(amide), a poly(ester), a poly(methylmethacrylate), a poly(ethylene), a poly(acrylonitrile), a poly(propylene), a thermoplastic poly(urethane), a poly(imide), a alkylene tetrafluoroethylene, a polycarbonate, a poly(ethylene oxide), a poly(caprolactone), or any copolymer or combination thereof.

88. The process of claim 85, wherein the at least one nanodiamond is characterized as having a characteristic cross-sectional dimension in the range of from about 1 nm to about 50 nm.

89. The process of claim 85, wherein the at least one nanodiamond is characterized as having a characteristic cross-sectional dimension in the range of from about 5 nm to about 20 nm.

90. The process of claim 85, wherein the weight percent of nanodiamonds in the mixture is in the range from about 0.01 percent to about 90 percent based on total weight of the mixture

91. The process of claim 85, wherein the weight percent of nanodiamonds in the mixture is in the range from about 10 percent to about 60 percent based on total weight of the mixture.

92. The process of claim 85, wherein the weight percent of nanodiamonds in the mixture is in the range from about 15 percent to about 30 percent based on total weight of the mixture.

93. The process of claim 85, wherein the at least one nanodiamond is produced by detonation synthesis.

94. The process of claim 85, wherein the at least one nanodiamond is a functionalized nanodiamond.

95. The process of claim 85, wherein the at least one nanodiamond is covalently bonded to the polymer.

96. The process of claim 85, further comprising fusing the polymer and the at least one nanodiamond.

97. An article made according to the process of claim 85.
